(19) United States
(12) Patent Application Publication
(10) Pub. No.: US 2026/0232815 A1
(43) Pub. Date: Aug. 13, 2026

(54) COPOLYMER-DRUG CONJUGATE FOR TREATMENT OF TUMOURS

(71) Applicant: Danmarks Tekniske Universitet, Kongens Lyngby (DK)

(72) Inventors: Anton Allen Abbotsford Autzen, Kongens Lyngby (DK); Neil Jean Borthwick, Kongens Lyngby (DK)

(21) Appl. No.: 19/154,167

(22) PCT Filed: Feb. 16, 2024
(86) PCT No.: PCT/EP2024/053994

§ 371 Date: Aug. 6, 2025

(30) Foreign Application Priority Data
Feb. 17, 2023 (EP) 23157339.5

(51) Int. Cl. A61K 47/58; A61K 39/385; A61P 35/00

(52) U.S. Cl. CPC A61K 47/58; A61K 39/385; A61P 35/00

(57) ABSTRACT

The present disclosure relates to a copolymer-drug-conjugate. Specifically disclosed herein are copolymer-drug-conjugates having good tissue retention. Disclosed herein are also copolymer-drug-conjugates for use in medicine, such as for use in the treatment of cancers. Disclosed herein are also methods for producing such copolymer-drug-conjugates.

### TECHNICAL FIELD

The present invention relates to a conjugate between a copolymer and an anti-cancer drug. It also relates to the method of treatment and imaging of solid cancer tumours with the composition thereof.

### BACKGROUND

Cancer immunotherapy has held out the promise of harnessing a patient's own T cells to attack cancer cells. The immune system's natural capacity to detect and destroy abnormal cells may prevent the development of many cancers.

However, cancer cells are sometimes able to avoid detection and destruction by the immune system. Cancer cells can reduce the expression of tumour antigens on their surface, making it harder for the immune system to detect them; express proteins on their surface that induce immune cell inactivation; and/or induce cells in the microenvironment to release substances that suppress immune responses and promote tumour cell proliferation and survival. While the features of solid tumours vary considerably, some physiological properties are common and create a characteristic microenvironment for tumour growth and proliferation.

Toll-like receptors (TLRs) are a class of pattern recognition receptors that play a bridging role in innate immunity and adaptive immunity. The activation of TLRs induces inflammatory responses and contributes to the development of antigen-specific anti-cancer immunity. TLR7 and TLR8, canonical members of the TLR family, are intracellular receptors expressed on the membrane of endosomes. TLR7 and TRL8 can be triggered not only by single-stranded RNA during viral infections, but also by immune modifiers that structurally mimic nucleosides. One example of artificial TLR agonist is imiquimod, which was first clinically approved for topical administration in 1997. Since then, new iterations of TLR7 and TLR8 agonists have been developed, including resiquimod (R848), motolimod (VTX-2337), and others, which offer improved solubility and more pronounced downstream affects.

Despite the emerged clinical interest and promising results in clinical studies, their clinical translation has been limited due to a narrow therapeutic window, motivated by a high systemic toxicity, limited in vivo residence and difficult production. Improving the tolerability and efficacy of such agonists using chemical modifications to localize them in the tumour microenvironment can broaden their clinical applicability.

Developing a vaccine strategy capable of inducing robust anti-tumour immunity concurrent with minimal systemic side effects, while inducing a prolonged and highly-localised tumour-specific effect is crucial for the continued progress of TLR7/8 agonist-based cancer immunotherapies towards widespread clinical translation.

### SUMMARY

Disclosed herein are copolymer-drug-conjugates. The copolymer of the disclosure provides a useful platform for the delivery of drugs to tissues. When administered to a tissue, for example by injection, the copolymer-drug-conjugate is retained in said tissue for an extended period of time. This retention is imparted by the properties of the copolymer.

The long tissue retention is useful for the delivery of drugs that must act locally on tissues. For example, said drug may on its own exhibit systemic toxicity; conjugation to the copolymer of the disclosure then reduces the systemic exposure of the drug, as it is retained locally where the conjugate is administered. The improved tissue retention also extends the exposure of the tissue to the drug, which can enhance the effect of the administered drug on said tissue. The improved retention also effects that the drug may be metabolised and/or secreted at a much slower rate, than had the drug been administered in isolation.

The copolymer of the present disclosure is furthermore amphiphilic, which enables internalisation of the conjugate by penetration of cell membranes. Thus, drug moieties conjugated to the copolymer may assert their effect on either extracellular or intracellular targets.

In particular, the copolymer of the disclosure provides for a drug delivery platform that is useful in the treatment of solid tumours through the consistent activation of immune cells. The platform is especially suited for TLR 7/8 agonists, while limiting off-target effects, and systemic side effects. Furthermore, by conjugation of diagnostic dyes to the copolymer, the copolymer-drug-conjugate of the disclosure allows in vivo imaging, such as imaging of solid tumours.

One aspect of the present disclosure provides a copolymer-drug-conjugate comprising an amphiphilic copolymer and 3 or more anti-cancer drug moieties conjugated to said amphiphilic copolymer. In a specific aspect of the disclosure, the copolymer is a poly(acrylic acid-co-styrene) copolymer, a poly(methacrylic acid-co-styrene) copolymer, a poly(maleic acid-co-styrene) copolymer, or a poly(diisobutylene-co-maleic acid) copolymer.

One aspect of the disclosure provides for a copolymer-drug-conjugate having the structure of formula (III):

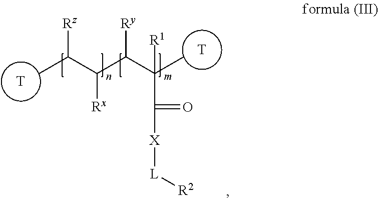

wherein:
each R¹ is independently selected from CH₃ and H,
each R^{y} is independently selected from H, COOH, and COX-L-R²,
each R^{z} is independently selected from H and CH₃,
each R^{x} is independently selected from:

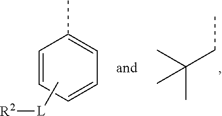

each X is independently selected from O, S, or NH,
each L is independently a bond or a linking group,
each R² is independently selected from H or an anti-cancer drug, wherein at least 3 of R² are an anti-cancer drug moiety,
each T is the same or different terminal groups,
n is between 5 and 500,
m is between 5 and 500,
wherein the copolymer is a random, alternating, or statistical copolymer.

One aspect of the present disclosure provides for a copolymer-drug-conjugate having the structure of formula (I):

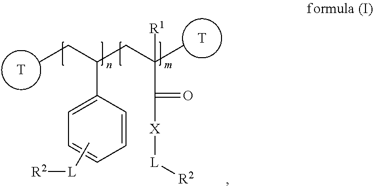

wherein:
each R¹ is independently selected from CH₃ and H,
each X is independently selected from O, S, or NH,
each L is independently a bond or a linking group,
each R² is independently selected from H or an anti-cancer drug, wherein at least 3 of R² are an anti-cancer drug moiety,
each T is the same or different terminal groups,
n is between 5 and 500,
m is between 5 and 500,
wherein the copolymer is a random, alternating, or statistical copolymer.

The synthesis method of the present disclosure enables facile functionalisation of the poly(acrylic acid-co-styrene) (AASTY) co-polymer without altering its original properties. Chemical handle moieties are present in the AASTY co-polymer that enable the insertion of, for example a Cyanine 7 near-infrared dye, for imaging purposes, via reaction with of azide moiety on the R terminus of AASTY_{9.9}-N₃. Furthermore, the pendant groups of the AASTY co-polymer, the acrylic acid groups, enable the conjugation of a TLR7/8 agonist to achieve anti-tumour in vivo effects. Thus, one aspect of the present disclosure provides for a method for synthesising the copolymer-drug-conjugate of the disclosure, said method comprising conjugating a dye or a drug, such as an anti-cancer drug, to a precursor poly(methacrylic acid-co-styrene) copolymer or a precursor poly(acrylic acid-co-styrene) copolymer. Such precursor copolymers may have (pendant) reactive handles.

One aspect of the present disclosure provides for a pharmaceutical composition comprising the copolymer-drug-conjugate of the disclosure.

One aspect of the disclosure provides for the copolymer-drug-conjugate of the disclosure or the pharmaceutical composition of the disclosure, for use in medicine.

The copolymer-drug-conjugate of the disclosure reverses tumour growth and increases the overall survival rate after tumour inoculation. Furthermore, the copolymer-drug-conjugate of the disclosure induces an adaptive response, since re-inoculation of a tumour after treatment did not result in tumour growth.

One embodiment of the disclosure provides for a use of a copolymer polymerized from:
a. styrene and/or diisobutylene, and
b. acrylic acid, methacrylic acid, and/or maleic acid
   for retaining an agent within a tissue. In one embodiment, the copolymer is a poly(acrylic acid-co-styrene) copolymer, a poly(methacrylic acid-co-styrene) copolymer, a poly(maleic acid-co-styrene) copolymer, or a poly(diisobutylene-co-maleic acid) copolymer.

The copolymer-drug-conjugate of the disclosure shows strong adherence to the tissue where the injection is performed with low diffusing to other tissues over time.

### DESCRIPTION OF DRAWINGS

FIG. 1. A: General synthetic scheme for the chemical preparation of the AASTY copolymer by reaction between styrene and acrylic acid, in the presence of RAFT polymerization agent. The subsequent product is then functionalised with a TLR agonist and/or an imaging dye. B: Chart showing the in vitro cellular response towards various percentages (0%, 2.5%, 6.5% and 10%) of conjugated TLR7/8a agonist. A direct regression between the observed cellular response and the amount of conjugated TLR7/8a agonist is observed, with a maximum signal at 10% of conjugated agonist.

FIG. 2. A: Fluorescence imaging study in two sets of five mice replicates, after respective subcutaneous injections of the free CY7 dye and AASTY-CY7. For both injections, most of the fluorescence signal remains in the near area of the initial injection depot and does not significantly diffuse away. While the integrated signal density slowly decreased over time, a large portion of the AASTY-CY7 signal was still clearly visible after 2 weeks, with an unchanged distribution; unlike for the free Cy7 control, which was significantly cleared from the injection site during the first few hours. B: Fluorescence signal at the isolated organs after respective tail intravenous injections of the free CY7 and AASTY-CY7. For both compounds, and the negative control, the signal of the region on interest (ROI) is highest in the injection site (the tail) and the liver, thus further supporting slow clearance via hepatic route.

FIG. 3. A: Overview of the timeline of tumour inoculation in mice, followed by treatment and tumour re-injection, to evaluate induced resistance. B: Graphic showing exponential tumour growth for the six mice replicates, without treatment. All individuals died in less than 25 days. C: Graphic showing the tumour growth timeline for the six mice replicates treated with the free TLR7/8a agonist. Minor improvement in tumour treatment efficacy is seen compared to the control; two out of six mice recovered, but the other four did not survive. D: Graphic showing the tumour growth timeline for the six mice replicates treated with the AASTY copolymer alone. Although AASTY delayed tumour growth for one replicate, no mice achieved eventual full recovery E: Graphic showing the tumour growth timeline for the six mice replicates treated with the AAASTY-TRL7/8a 1× (low dose). Tumours grew exponentially for five mice, whereas the remaining one experimented tumour size decrease and eventual recovery. F: Graphic showing the tumour growth timeline for the six mice replicates treated with the AAASTY-TRL7/8a 2× (high dose). Absence of tumour growth is seen for the first 20 days, and an overall negative tumour growth rate in the first 10 days. Eventually, four mice recovered from the tumour. G: Overview of the mice survival rates after tumour inoculation with the different treatments: free TLR7/8a, AASTY, AASTY-TLR7/8a 1× (low dose), AASTY-TLR7/8a 2× (high dose), negative control. AASTY-TLR7/8a 2× achieved the best result, since 80% of the mice survived after 60 days from tumour inoculation. H: Time evolution of the averaged tumour volumes with the different treatments: free TLR7/8a, AASTY, AASTY-TLR7/8a 1× (low dose), AASTY-TLR7/8a 2× (high dose), negative control. AASTY-TLR7/8a 2× (high dose) achieves the best outcome, since the tumour volume steadily decreases during 10 days. I: Mean survival time after mice tumour injection for the with the different treatments: free TLR7/8a, AASTY, AASTY-TLR7/8a 1× (low dose), AASTY-TLR7/8a 2× (high dose), negative control. The highest survival time, above 100 days, is achieved with AASTY-TLR7/8a 2× (high dose). J: Averaged slope coefficient for the tumour growth plots. Whereas the tumour growth rate is positive for the control, free TLR7/8a, AASTY and AASTY-TLR7/8a 1× (low dose); AASTY-TLR7/8a 2× (high dose) achieves a negative rate, thus indicating anti-tumour effect. K: Graphic showing the tumour growth timeline for the six mice replicates, after tumour re-inoculation. After the inoculation, no tumour growth was observed for those individuals treated with AASTY-TLR7/8a 1× (low dose) and AASTY-TLR7/8a 2× (high dose). L: Tumour imaging showing the biodistribution of AASTY-TLR7/8a-CY7 2× (high dose) after intra tumour administration. The data shows an extremely long residence time for the AASTY-TLR7/8a-CY7 2× (high dose), with a half-life estimated at 41 days and most of the signal still clearly visible when the study was terminated after 117 days (acquisition week 15). M: 3D model showing the biodistribution of the AASTY-TLR7/8a-CY7 2× (high dose) based on the fluorescence tomography scans performed 5 to 6 days after single dose administration. AASTY-TLR7/8a-CY7 diffuses through the bean-shaped tumour (with a signal maximum at the injection point) and primarily accumulates in the liver.

FIG. 4: Embodiment of the present disclosure consisting of a copolymer-drug-conjugate having a number of styrene moieties, a number of free acrylic acid moieties, a number of acrylic acid moieties conjugated to a drug moiety “R”, and terminal groups obtained from RAFT polymerisation.

FIG. 5: 3D reconstructions of a fluorescence tomography imaging study in two sets of 3 mice replicates, after respective subcutaneous injections of AASTY-Cy7 and MAASTY-Cy7. Both conjugates exhibit a similar biodistribution pattern with most of the fluorescence signal remaining in the near area of the initial injection depot and a small fraction spreading to the lymphatic network over time. After 5 days, a large portion of the Cy7 signal was still clearly visible for both conjugates, suggesting that the two polymers have similar pharmacokinetic profiles. High fluorescence appears as solid grey, whereas low fluorescence appears as diffuse clouds of grey.

FIG. 6: Comparison of structures of AASTY, MAASTY, SMA, and DIBMA. Terminal groups according to the disclosure are not shown in the structures. All copolymers are comprised of both hydrophobic and hydrophilic monomers.

### DETAILED DESCRIPTION

### Definitions

The term “copolymer” is one of the art. It refers to a polymer comprising two or more different monomer units that are polymerized in a process called copolymerization. Since a copolymer comprises at least two different monomer units, copolymers can be classified based on how the monomer units are arranged to form a polymer chain. Those classifications include “alternating copolymers” (in which the monomers units repeat with a highly regular alternating pattern), “periodic copolymers” (in which the monomers units are arranged with a repeating sequence), “statistical copolymers” (in which the sequence of monomer units follows a statistical rule), “random copolymers” (in which the monomer units are attached in a random order), and “block copolymers” (in which two or more homopolymer subunits are linked).

By “amphiphilic copolymer” is meant a copolymer comprising or consisting of both hydrophobic and hydrophilic monomers. The amphiphilic copolymer of the disclosure is preferably a random, an alternating, or a statistical copolymer with respect to the hydrophobic and hydrophilic monomers. The amphiphilic copolymer of the disclosure is preferably not a block copolymer.

By “methacrylic acid” is meant the compound having the CAS registry number 79-41-4. By “acrylic acid” is meant the compound having the CAS registry number 79-10-7. By “styrene” is meant the compound having the CAS registry number 100-42-5. By “maleic acid” is meant the compound having the CAS registry number 110-16-7. By “diisobutylene” is meant the compound having the CAS registry number 107-39-1.

By “copolymer-drug-conjugate” is meant a molecular structure composed of a copolymer chemically linked, through a bond, a spacer, or linker group, to a molecule (other than food) that is used to prevent, diagnose, treat, or relieve symptoms of a disease or abnormal condition, wherein the condition is cancer. As used herein, a “spacer” or “linker group” is a chemical entity that connects two or more molecules.

By “drug” is meant a compound or moiety that is used to prevent, diagnose, treat, ameliorate, or relieve symptoms of a disease or abnormal condition.

The term “PEG” as used herein refers to an ethylene glycol polymer of variable length, typically with relative molecular weights of 200-8000 Da.

The term “TLR” as used herein refers to single-pass membrane-spanning receptors usually expressed by cells such as macrophages. Activation of TLR receptors activate immune cell responses.

As used herein, “solid tumour” refers a mass of abnormally growing cells that affects solid tissues and/or organs such as the breast or prostate, as opposed to leukemia, a cancer affecting the blood, which is a fluid tissue.

The term “dye” as used herein refers to a chemical substance that is coloured and chemically bonds to the substrate to which it is being applied. As used herein, the term “fluorescent dye” refers to a compound or moiety capable of both absorbing and emitting electromagnetic radiation, for example in the visible, IR, or UV region of the spectrum of electromagnetic radiation.

As used herein, “cyanine dye” refers to closed-chain cyanine dyes, i.e., cyanine dyes having end groups that are cyclic moieties, wherein the cyclic moieties may be aromatic or non-aromatic and substituted or unsubstituted at one or more positions. The term “cyanine dyes” or “cyanines” refers to any of the cyanine dyes that are well known in the art. Synthetic approaches have been disclosed in EP 1,065,250; WO 05/014723; WO 99/31181; U.S. Pat. Nos. 5,268,486; 5,658,751; 5,808,044; 5,981,747; 5,658,751; 4,937,198; 4,937,198; 6,080,868; 6,110,630; 6,225,050; 6,238,838; 6,716,994 and 6,207,464, as well as U.S Publication No. 2003/0113755.

As used herein, “triarylmethane dye” refers to a dye, which is integrated by a backbone containing three phenyl rings connected to a common carbon atom, wherein the phenyl rings are unsubstituted or substituted at one or more positions with identical or different substituents.

By “indocyanine green” is meant the compound having the CAS registry number 3599-32-4.

By “cyanine 7” is meant the compound having the CAS registry number 477908-53-5.

By “fluorescein” is meant the compound having the CAS registry number 2321-07-5.

By “thiazine dye” is meant a dye integrated by at least a thiazine ring, wherein thiazine is substituted at one or more positions with identical or different substituents.

By “methylene blue” is meant the compound having the CAS registry number 61-73-4.

The term “intratumoral injection” as used herein means the direct injection of agents into the tumor, wherein the agents are a copolymer-drug conjugate, such as AASTY-TLR7/8a construct. The intratumoral injection is performed onto the living organism subjected to this study, wherein subject has been inoculated with a colon carcinoma.

As used herein, a “reactive handle” is a moiety or functional group on a compound capable of reacting with a moiety or functional group, such as selectively reacting with such. Examples hereof are a carboxylic acid or an amine (forming amides); or an alkyne or an azide (forming a triazole); or a haloalkyl group capable of reacting via an S_{N}1 or an S_{N}2 reaction.

The term “immunostimulator” as used herein means a substance that stimulate the immune system by inducing activation or increasing activity of any of its components.

### Copolymer-Drug-Conjugate of the Disclosure

The copolymer of the present disclosure is a synthetic AASTY copolymer. AASTY copolymers feature an increased tissue residence time, for example after sub-cutaneous injection. The increase residence time in tissues is to be seen in comparison to injecting/administering the drug without conjugation to the copolymer of the disclosure. However, the copolymer-drug-conjugate of the disclosure may also exhibit improved tissue retention in comparison to other polymer-drug-conjugates. Combining the good tissue retention property of the copolymer with covalently attached therapeutically active moieties, such as anti-cancer agents, such as TLR 7/8 agonists, can for example improve activation of immune cells at the site of injection, enabling the use of TLR 7/8 agonists in the treatment of solid tumours, while limiting off-target effects, and side effects linked to systemic exposure.

One embodiment of the disclosure provides for a copolymer-drug-conjugate comprising an amphiphilic copolymer and one or more anti-cancer drug moieties conjugated to said amphiphilic copolymer. In a preferred embodiment, the copolymer-drug-conjugate comprises two or more anti-cancer drug moieties. In an even more preferred embodiment, the copolymer-drug-conjugate comprises three or more anti-cancer drug moieties. In one embodiment, the anti-cancer drug moieties are conjugated to said amphiphilic copolymer via a bond or a linking group.

In one embodiment, the amphiphilic copolymer is an anionic amphiphilic copolymer. Anionic amphiphilic copolymer can for example be those having carboxylic acid moieties incorporated.

In one embodiment, the amphiphilic copolymer is a copolymer of:
1. styrene and/or diisobutylene, and
2. acrylic acid, methacrylic acid, and/or maleic acid.

In a preferred embodiment of the disclosure, the amphiphilic copolymer is a poly(acrylic acid-co-styrene) copolymer, a poly(methacrylic acid-co-styrene) copolymer, a poly(maleic acid-co-styrene) copolymer, or a poly(diisobutylene-co-maleic acid) copolymer. In an even more preferred embodiment of the disclosure, the amphiphilic copolymer is a poly(acrylic acid-co-styrene) copolymer or a poly(methacrylic acid-co-styrene) copolymer.

One embodiment of the disclosure provides for a copolymer-drug-conjugate having the structure of formula (III):

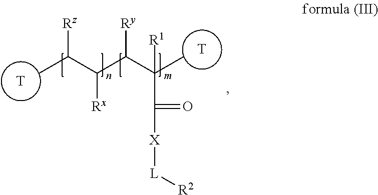

wherein:
each R¹ is independently selected from CH₃ and H,
each R^{y} is independently selected from H, COOH, and COX-L-R²,
each R^{z} is independently selected from H and CH₃,
each R^{x} is independently selected from

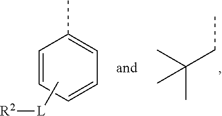

each X is independently selected from O, S, or NH,
each L is independently a bond or a linking group,
each R² is independently selected from H or an anti-cancer drug, wherein at least 3 of R² are an anti-cancer drug moiety,
each T is the same or different terminal groups,
n is between 5 and 500,
m is between 5 and 500,
wherein the copolymer is a random, alternating, or statistical copolymer.

In one embodiment R^{x} is

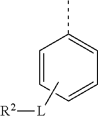

### and R^{z} is H.

In one embodiment R^{x} is

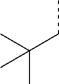

and R^{z} is CH₃.

In one embodiment R¹ is H and R^{y} is COOH or COX-L-R².

In one embodiment R¹ is H and R^{y} is H.

In one embodiment R¹ is CH₃ and R^{y} is H.

In a specific embodiment of the disclosure R^{x} is

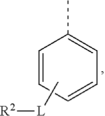

R^{z} is H, R¹ is H, and R^{y} is COOH or COX-L-R².

In a specific embodiment of the disclosure R^{x} is

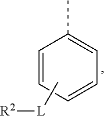

R^{z} is H, R¹ is H, and R^{y} is H.

In a specific embodiment of the disclosure R^{x} is

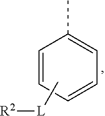

R^{z} is H, R¹ is CH₃, and R^{y} is H.

In a specific embodiment of the disclosure R^{x} is

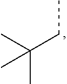

R^{z} is CH₃, R¹ is H, and R^{y} is COOH or COX-L-R².

One embodiment of the present disclosure provides for a copolymer-drug-conjugate having the structure of formula (I):

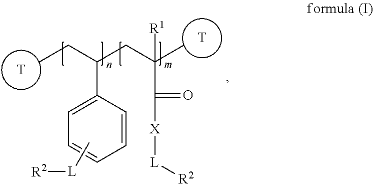

wherein:
each R¹ is independently selected from CH₃ and H,
each X is independently selected from O, S, or NH,
each L is independently a bond or a linking group,
each R² is independently selected from H or an anti-cancer drug, wherein at least 3 of R² are an anti-cancer drug moiety,
each T is the same or different terminal groups,
n is between 5 and 500,
m is between 5 and 500,
wherein the copolymer is a random, alternating, or statistical copolymer.

In one embodiment, R¹ is CH₃. In one embodiment, R¹ is H. In one embodiment, the copolymer-drug-conjugate of the present disclosure is a poly(methacrylic acid-co-styrene) copolymer or a poly(acrylic acid-co-styrene) copolymer.

In one embodiment the copolymer-drug-conjugate has the structure of formula (II):

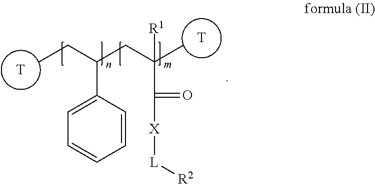

In one embodiment, X is selected from O, S, or NH, such as wherein X is O, such as wherein X is NH. When X corresponds to O, the functional group linking the copolymer and the anti-cancer drug is an ester. When X corresponds to NH, the functional group linking the copolymer and the anti-cancer drug is an amide.

In one embodiment, L is a bond. In one embodiment, L is a linking group selected from PEG, alkylenes, triazoles, and a group form from reaction between a haloalkyl and a nucleophile.

In one embodiment, n and m are each between 5 and 10, between 10 and 15, between 15 and 20, between 20 and 30, between 30 and 40, between 40 and 50, between 50 and 60, between 60 and 70, between 70 and 80, between 80 and 90, between 90 and 100, between 100 and 120, between 120 and 150, between 150 and 200, between 200 and 250, between 250 and 300, between 300 and 350, between 350 and 400, between 400 and 450, and/or between 450 and 500. In one embodiment, n is between 5 and 10, between 10 and 15, between 15 and 20, between 20 and 30, between 30 and 40, between 40 and 50, between 50 and 60, between 60 and 70, between 70 and 80, between 80 and 90, such as between 90 and 100, such as between 100 and 120, such as between 120 and 150, such as between 150 and 200, such as between 200 and 250, such as between 250 and 300, such as between 300 and 350, such as between 350 and 400, such as between 400 and 450, such as between 450 and 500. In one embodiment, m is between 5 and 10, between 10 and 15, between 15 and 20, between 20 and 30, between 30 and 40, between 40 and 50, between 50 and 60, between 60 and 70, between 70 and 80, between 80 and 90, such as between 90 and 100, such as between 100 and 120, such as between 120 and 150, such as between 150 and 200, such as between 200 and 250, such as between 250 and 300, such as between 300 and 350, such as between 350 and 400, such as between 400 and 450, such as between 450 and 500. In one embodiment of the disclosure, the ratio nim is between 10:1 and 1:10. In one embodiment of the disclosure, the ratio nim is between 10:1 and 9:1, between 9:1 and 8:1, between 8:1 and 7:1, between 7:1 and 6:1, between 6:1 and 5:1, between 5:1 and 4:1, between 4:1 and 3:1, between 3:1 and 2:1, between 2:1 and 3:2, between 3:2 and 1:1, between 1:1 and 2:3, between 2:3 and 1:2, between 1:2 and 1:3, between 1:3 and 1:4, between 1:4 and 1:5, between 1:5 and 1:6, between 1:6 and 1:7, between 1:7 and 1:8, between 1:8 and 1:9, and/or between 1:9 and 1:10.

In one embodiment of the present disclosure, the copolymer has a molecular mass of less than 100 kDa, such as less than 90 kDa, such as less than 80 kDa, such as less than 70 kDa, such as less than 60 kDa, such as less than 50 kDa, such as less than 40 kDa, such as less than 30 kDa, such as less than 20 kDa, such as less than 15 kDa, such as less than 14 kDa, such as less than 13 kDa. In one embodiment, the copolymer has a molecular mass of at least 1 kDa, such as at least 3 kDa, such as at least 5 kDa, such as at least 7 kDa, such as at least 9 kDa. In one embodiment of the disclosure, the copolymer has a molecular weight of 1 to 100 kDa, such as 1 to 5 kDa, such as 5 to 9 kDa, such as 9 to 13 kDa, such as 13 to 17 kDa, such as 17 to 20 kDa, such as 20 to 30 kDa, such as 30 to 40 kDa, such as 40 to 50 kDa, such as 50 to 60 kDa, such as 60 to 70 kDa, such as 70 to 80 kDa, such as 80 to 90 kDa, such as 90 to 100 kDa. In one embodiment, the copolymer has a molecular weight of 1 to 5 kDa, 5 to 9 kDa, 9 to 13 kDa, 13 to 17 kDa, 17 to 20 kDa, 20 to 30 kDa, 30 to 40 kDa, 40 to 50 kDa, 50 to 60 kDa, 60 to 70 kDa, 70 to 80 kDa, 80 to 90 kDa, and/or 90 to 100 kDa The mass of the copolymer is preferably assessed disregarding the masse of any drug and/or terminal group conjugated thereto. Specifically, when referring to the mass of the copolymer, reference is made to the structure of formula (I) without the moieties T, L and/or R².

In one embodiment of the present disclosure, the anti-cancer drug is an anti-tumour drug. As shown herein in the examples, the carboxylic acid groups of the presently disclosed copolymers were used to conjugate a TLR7/8 agonist to trigger immune responses in vivo. The copolymer-drug-conjugate shown in the examples remained soluble in water, having a substantial amount of its carboxylic acid groups conjugated to the test drug. This was observed despite a high content of styrene in the copolymer. In one embodiment of the disclosure, the anti-tumour drug is an anti-tumour drug with effect against solid tumours. In one embodiment of the present disclosure, the anti-cancer drug is a Toll-Like Receptor (TLR) agonist, such as a TLR7 agonist, a TLR8 agonist, or a TLR7/8 agonist. Toll-like receptor agonists, such as TLR7/8, stimulate immune response against cancer cells.

In one embodiment, at least 3 of R² are an anti-cancer drug moiety, such as at least 4, at least 5, at least 6, at least 7, at least 8, at least 9 or at least 10 of R². In one embodiment, 2% of the monomeric units in the copolymer are conjugated to an anti-cancer drug moiety, such as at least 3%, at least 4%, at least 5%, at least 6%, at least 7%, at least 8%, at least 9%, or at least 10%.

In one embodiment of the present disclosure, anti-cancer drug has the structure of formula (A-I), formula (A-II), formula (A-III), or formula (A-IV):

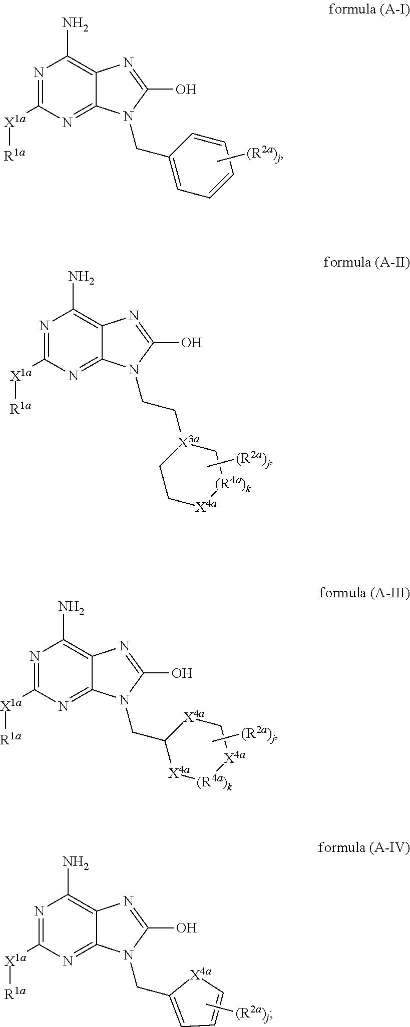

wherein
X^{1a} is —O—, —S—, or —NR^{Ca};
R^{1a} is hydrogen, (C₁₋₁₀)alkyl, substituted (C₁₋₁₀)alkyl, C₆₋₁₀aryl, or substituted C₆₋₁₀aryl, C₅₋₉heterocyclic, substituted C₅₋₉heterocyclic;
R^{Ca} is hydrogen, (C₁₋₁₀ alkyl, or substituted C₁₋₁₀alkyl; or R^{Ca} and R^{1a} taken together with the nitrogen atom to which they are attached form a heterocyclic ring or a substituted heterocyclic ring;
each R^{2a} is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted-C(O)—(C₁-C₆)alkyl, —C(O)—(C₆-C₁₀) aryl (aroyl), substituted-C(O)—(C₆-C₁₀) aryl, —C(O)OH (carboxyl), —C(O)O(C₁-C₆)alkyl (alkoxycarbonyl), substituted-C(O)O(C₁-C₆)alkyl, —NR^{aa}R^{ba}, —C(O) NR^{aa}R^{ba} (carbamoyl), halo, nitro, or cyano, or R^{2a} is absent;
each R^{aa} and R^{ba} is independently hydrogen, (C₁-C₆)alkyl, substituted (C₁-C₆)alkyl, (C₃-C₅) cycloalkyl, substituted (C₃-C₈) cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₅)alkoxycarbonyl;
wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxy C₁₋₆alkylene, amino, cyano, halo, or aryl;
j is 0, 1, 2, 3 or 4;
X^{3a} is —N— or —CH—;
R^{4a} is —CH₂— or —CH(R^{2a})—;
k is 0 or 1;
X^{4a} is —O—, —S—, —NH—, —N(R^{da})—, —CH₂—, or —CH(R^{2a})—;
each R^{da} is independently-OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted-C(O)—(C₁-C₆)alkyl, —C(O)—(C₆-C₁₀) aryl (aroyl), substituted-C(O)—(C₆-C₁₀) aryl, —C(O)O(C₁-C₆)alkyl (alkoxycarbonyl), substituted-C(O)O(C₁-C₆)alkyl, —C(O) NR^{aa}R^{ba} (carbamoyl); or a tautomer thereof.

In one embodiment of the present disclosure, the anti-cancer drug has the structure of formula (B-I):

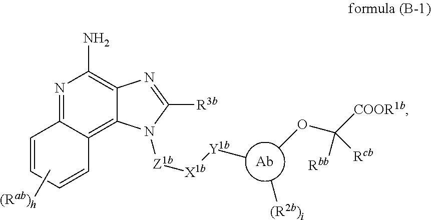

wherein
R^{1b} represents (C₁₋₈)alkyl, (C₃₋₈) cycloalkyl, or a 3- to 8-membered saturated heterocyclic ring group comprising a O atom, wherein R^{1b} is optionally substituted by one or more substituents independently selected from halogen, cyano, hydroxyl and (C₁₋₃)alkoxy;
Z^{1b} represents a (C₂₋₆)alkylene group, wherein a carbon atom in Z^{1b} which is not adjacent to a nitrogen atom may be replaced with an oxygen atom;
X^{1b} represents NR^{5b}, >N—COR^{5b}, >N—CONR^{5b}R^{5ab}, CONR^{5b}, NR^{5b}CO, NR^{5b}CONR^{6b} or NR^{6b}CONR^{5b};
Y^{1b} represents a single bond or (C₁₋₆)alkylene;
each R^{2b} is independently selected from halogen, cyano, hydroxy, thiol, (C₁₋₃)alkyl, (C₁₋₃) hydroxyalkyl, (C₁₋₃)haloalkyl, (C₁₋₃)alkoxy, (C₁₋₃)haloalkoxy, (C₁₋₃)alkylthio, (C₁₋₃)alkylsulfonyl and (C₁₋₃)alkylsulfinyl;
R^{3b} represents C₁₋₆ alkyl optionally substituted by (C₁₋₆)alkoxy;
each R^{ab} is independently selected from halogen, cyano, hydroxy, thiol, (C₁₋₃)alkyl, (C₁₋₃) hydroxyalkyl, (C₁₋₃)haloalkyl, (C₁₋₃)alkoxy, (C₁₋₃)haloalkoxy, (C₁₋₃)alkylthio, (C₁₋₃)alkylsulfonyl and (C₁₋₃)alkylsulfinyl;
R^{5b} and R^{5ab} each independently represents hydrogen, a 3- to 8-membered saturated heterocyclic ring comprising a ring group O, S(O)ₚ or NR^{10b}, a (C₁₋₆)alkyl group or (C₃₋₆) cycloalkyl group, the latter two groups being optionally substituted by one or more substituents independently selected from NR^{7b}R^{8b} or R^{9b}, R^{7b} and R^{8b} each independently represent hydrogen, a 3- to 8-membered saturated heterocyclic ring comprising a ring group O, S(O)ₚ or NR^{10ab}, (C₁₋₆)alkyl or (C₃₋₆) cycloalkyl, the latter two groups being optionally substituted by one or more groups independently selected from halogen, cyano, S(O)_{q}R^{11b}, OR^{12b}, CO₂R^{12b}, OC(O) R^{12b}, SO₂NR^{12b}R^{13b} CONR^{12b}R^{13b}, NR^{12b}R^{13b}, NR^{12b}SO₂R^{14b}, NR^{12b}COR^{13b}, or a 3- to 8-membered saturated heterocyclic ring comprising a ring group O, S(O)ₚ or NR^{10bb},
or R^{7b} and R^{8b} together with the nitrogen atom to which they are attached form a 3- to 8-membered saturated heterocyclic ring comprising a ring nitrogen atom and optionally one or more further heteroatoms independently selected from nitrogen, oxygen, sulphur and sulphonyl, the heterocyclic ring being optionally substituted by one or more substituents independently selected from halogen, cyano, S(O)_{q}R^{15b}, OR^{15b}, CO₂R^{15b}, COR^{15b}, OC(O) R^{15b}, SO₂NR^{15b}R^{16b}, CONR^{15b}R^{16b}, NR^{15b}R^{16b}, NR^{15b}SO₂R^{17b}, NR^{15b}COR^{16b}, NR^{15b}CO₂R^{16b}, heteroaryl, (C₁₋₆)haloalkyl, (C₃₋₈) cycloalkyl and (C₁₋₆)alkyl, the latter two groups being optionally substituted by one or more groups independently selected from cyano, S(O)_{q}R^{18b}, OR^{18b}, CO₂R^{18b}, SO₂NR^{18b}R^{19b}, CONR^{18b}R^{19b} or NR^{18b}R^{19b};
R^{Ob} represents halogen, cyano, CO₂R^{20b}, S(O)_{q}R^{20b}, OR^{20b}, SO₂NR^{20b}R^{22b}, CONR^{20b}R^{22b}, NR^{20b}SO₂R^{21b}, NR^{20b}CO₂R^{21b}, NR^{20b}COR^{22b} or a 3- to 8-membered saturated heterocyclic ring comprising a ring group NR^{10cb};
R^{10b}, R^{10ab}, R^{10bb} and R^{10cb} independently represent hydrogen, CO₂R^{23b}, S(O)_{q}R^{23b}, COR^{24b}, or a (C₁₋₆)alkyl, (C₂₋₆)alkenyl, (C₂₋₆)alkynyl or (C₃₋₈) cycloalkyl group, each of which may be optionally substituted by one or more substituents independently selected from halogen, cyano, OR^{25b} or NR^{25b}R^{26b};
R^{6b}, R^{11b}, R^{12b}, R^{13b}, R^{15b}, R^{16b}, R^{18b}, R^{19b}, R^{20b}, R^{22b}, R^{24b}, R^{25b} and R^{26b} each independently represent hydrogen, (C₁₋₆)alkyl or (C₃₋₆) cycloalkyl;
R^{14b}, R^{17b}, R^{21b} and R^{23b} each independently represent (C₁₋₆)alkyl or (C₃₋₆) cycloalkyl;
h, i, p and q each independently represent an integer 0, 1 or 2; and
Ab represents a monocyclic or bicyclic (C₆₋₁₀) aryl or a monocyclic or bicyclic (C₅₋₁₂) heteroaryl group containing 1-3 heteroatoms; and
R^{bb} and R^{cb} independently represent hydrogen or (C₁₋₆)alkyl, or R^{bb} and R^{cb} combine together to form (C₃₋₈) cycloalkyl.

In one embodiment of the present disclosure, the anti-cancer drug has the structure of formula (C-I):

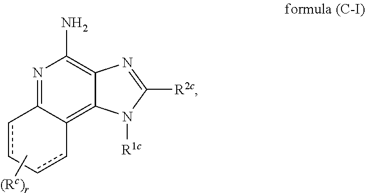

wherein
R^{1c} is selected from the group consisting of —(C₂₋₆)alkyl-N(R^{3c})₂, —(C₂₋₆)alkyl-NR^{3c}—SO₂—X^{c}—R^{4c}, and —(C₂₋₆)alkyl-NR^{8c}—SO₂—R^{7c};
X^{c} is a bond or —NR^{5c}—;
R^{4c} is alkyl, aryl, or heteroaryl;
R^{2c} is selected from the group consisting of hydrogen, alkyl, alkenyl, aryl, substituted aryl, heteroaryl, substituted heteroaryl, alkyl-O-aryl, alkyl-O-alkyl, alkyl-O-alkenyl, and alkyl or alkenyl substituted by one or more substituents selected from the group consisting of: OH, halogen, —N(R^{3c})₂, —CO—N(R^{3c})₂, —CO—(C₁₋₁₀)alkyl, —CO—O—(C₁₋₁₀)alkyl, —N₃, aryl, substituted aryl, heteroaryl, substituted heteroaryl, heterocyclyl, substituted heterocyclyl, —CO-aryl, —CO-(substituted aryl), —CO-heteroaryl, and —CO-(substituted heteroaryl);
each R^{3c} is independently selected from the group consisting of hydrogen and (C₁₋₁₀)alkyl,
R^{5c} is selected from the group consisting of hydrogen and (C₁₋₁₀)alkyl, or R^{4c} and R^{5c} can combine to form a 3 to 7 membered heterocyclic or substituted heterocyclic ring;
R^{6c} is selected from the group consisting of hydrogen and (C₁₋₁₀)alkyl;
R^{7c} is selected from the group consisting of hydrogen and (C₁₋₁₀)alkyl, or R^{6c} and R^{7c} can combine to form a 3 to 7 membered heterocyclic or substituted heterocyclic ring;
r is 0 to 4 and each Re present is independently selected from the group consisting of (C₁₋₁₀)alkyl, (C₁₋₁₀)alkoxy, halogen and trifluoromethyl.

In one embodiment of the present disclosure, anti-cancer drug is a TLR7, a TLR8, or a TLR7/8 agonist is selected from the list consisting of: Imiquimod, Resiquimod, Gardiquimod, 852A, Loxoribine, Bropirimine, 3M-011 (CAS no. 642473-62-9), 3M-052 (CAS no. 1359993-59-1), DSR-6434 (CAS no. 1059070-10-8), DSR-29133, SZU-101, SM-360320 (CAS no. 226907-52-4), SM-276001 (CAS no. 473930-22-2), VTX-2337 (CAS no. 926927-61-9), and 1-(3-aminopropyl)-2-(ethoxymethyl) imidazo[4,5-c]quinolin-4-amine. Shukla et al. 2011 is a SAR study of TLR7 agonists, which can be employed to predict where conjugation can be carried out without abrogating the activity of the agonist.

In one embodiment of the disclosure, the anti-cancer drug is a Stimulator of Interferon Genes (STING) agonist. In one embodiment, the anti-cancer drug is IL-2, IL-12, or IL-15, or a fragment thereof.

In one embodiment of the present disclosure, the anti-cancer drug is a radiopharmaceutical drug or radioactive agent.

In one embodiment, the copolymer-drug-conjugate comprises one type of anti-cancer drug. In one embodiment, the copolymer-drug-conjugate comprises two or more different anti-cancer drugs.

In one embodiment of the present disclosure, when R² is an anti-cancer drug, L is a linking group wherein the moiety R² is conjugated to L at a carbon atom or a heteroatom such as N, O, or S in R². In one embodiment of the present disclosure, when R² is an anti-cancer drug, L is a bond wherein the moiety R² is conjugated via bond L to X at a carbon atom or a heteroatom such as N, O, or S in R².

In one embodiment, T is independently selected from the group consisting of H, alkyl, substituted alkyl, nitrile, hydroxy, carboxyl, halogen, thiol, substituted thiol, acyl, substituted acyl, a group of formula X-I, a group of formula Y-I, a group of formula Y-II, a fluorescent dye, a diagnostic agent;
wherein the group of formula X-I has the structure:

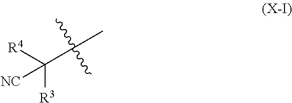

wherein R³ and R⁴ are each independently selected from hydrogen, alkyl, and substituted alkyl;
wherein the group of formula Y-I has the structure:

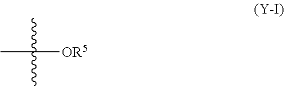

wherein R⁵ is selected from hydrogen, alkyl, and substituted alkyl; and
wherein the group of formula Y-II has the structure:

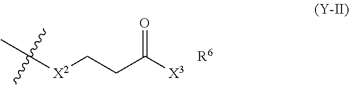

wherein R5 is selected from hydrogen, alkyl, and substituted alkyl, and
X² and X³ are each independently selected from S and O.

In one embodiment of the present disclosure, the fluorescent dye is selected from the group consisting of a cyanine dye such as indocyanine green or cyanine 7, a triarylmethane dye such as fluorescein, a thiazine dye such as methylene blue, LUM015, VGT-309, AVB-620, C-Dots, BLZ-100.

One embodiment of the present disclosure provides for a pharmaceutical composition comprises the copolymer-drug-conjugate of the disclosure. In one embodiment of the present disclosure the pharmaceutical composition is formulated for administration by injection, such as intratumoral injection.

### Synthetic Methods

In one embodiment of the present disclosure, the copolymer-drug-conjugate is synthesised by conjugating an anti-cancer drug to a precursor copolymer, such as a precursor poly(methacrylic acid-co-styrene) copolymer, a precursor poly(acrylic acid-co-styrene) copolymer, a precursor poly(maleic acid-co-styrene) copolymer, or a precursor poly(diisobutylene-co-maleic acid) copolymer.

In one embodiment of the present disclosure the precursor copolymer has the structure of formula (III-P):

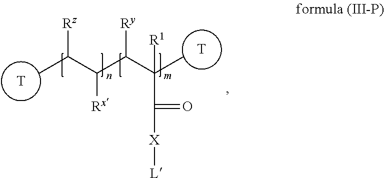

wherein R^{x′} is

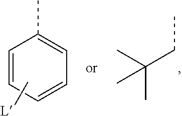

wherein each L′ is independently selected from H and a reactive handle, and
T, n, m, R¹, X, R^{z}, and R^{y} are as defined herein.

In one embodiment of the present disclosure the precursor poly(methacrylic acid-co-styrene) copolymer or the precursor poly(acrylic acid-co-styrene) copolymer has the structure of formula (I-P):

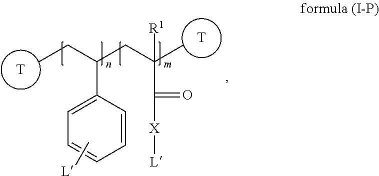

wherein each L′ is independently selected from H and a reactive handle, and
T, n, m, R¹, and X are as defined herein.

In one embodiment of the present disclosure, wherein each L′ is independently selected from the group consisting of H, CH₂Cl, CH₂Br, or CH₂I.

### Treatment

One embodiment of the disclosure provides for the copolymer-drug-conjugate of the disclosure or the pharmaceutical composition of the disclosure, for use in medicine. The copolymer of the present disclosure is amphiphilic, which enables internalisation of the conjugate by disruption of cell membranes. Thus, drug moieties conjugated to the copolymer may assert their effect on either extracellular or intracellular targets. One embodiment of the disclosure provides for the copolymer-drug-conjugate of the disclosure or the pharmaceutical composition of the present disclosure, for use in treatment of cancer.

One embodiment provides for the copolymer-drug-conjugate or the disclosure or the pharmaceutical composition of the disclosure for use in treatment of cancer. In one preferred embodiment, the cancer is characterised by the presence a tumour. In one embodiment, the tumour is a solid tumour. In one embodiment, the tumour, such as the solid tumour, is a sarcoma. In one embodiment, the tumour such as the solid tumour is a carcinoma. In one embodiment, the solid tumour is a primary or a metastatic tumour. or a secondary metastatic tumour.

In one embodiment the cancer is liver cancer or spleen cancer.

In one embodiment, the copolymer-drug-conjugate or the pharmaceutical composition is administered intratumourally, such as by injection.

One embodiment of the disclosure provides for a method of treating cancer, such as a cancer characterised by a tumour, such as a solid tumour, said method comprising administering the copolymer-drug-conjugate of the disclosure or the pharmaceutical composition of the present disclosure to a subject in need thereof.

One embodiment of the disclosure provides for a method of inducing an immune response in a subject, said method comprising administering the copolymer-drug-conjugate of the disclosure or the pharmaceutical composition of the disclosure to said subject.

One embodiment of the disclosure provides for a method of immunising a subject, said method comprising administering the copolymer-drug-conjugate according of the disclosure or the pharmaceutical composition of the disclosure to said subject.

One embodiment of the disclosure provides for a method of reducing tumour volume in a subject, said method comprising administering the copolymer-drug-conjugate of the disclosure or the pharmaceutical composition of the disclosure to said subject.

One embodiment of the present disclosure provides for a use of the copolymer-drug-conjugate of the disclosure or the pharmaceutical composition of the disclosure as an immunostimulator.

One embodiment of the present disclosure provides for a use of the copolymer-drug-conjugate of the present or the pharmaceutical composition of the disclosure for the manufacture of a medicament for use in the treatment of cancer.

One embodiment of the present disclosure provides for use of a poly(acrylic acid-co-styrene) or a poly(methacrylic acid-co-styrene) copolymer for retaining an agent. By “retaining an agent” is meant to increase the tissue retention of said compound/agent when administered to a subject, as a result of mixing, formulating or conjugating said compound/agent with a poly(acrylic acid-co-styrene) copolymer or a poly(methacrylic acid-co-styrene) copolymer. In one embodiment, the agent is as an anticancer agent, a diagnostic agent and/or a radiopharmaceutical agent. In one embodiment, the agent is the anti-cancer drug as disclosed herein. In one embodiment, the agent is an agent capable of inducing an immune response.

One embodiment provides for a method of increasing the residence time of an agent in a tissue, said method comprising administering the agent together with a copolymer of the disclosure. The tissue may be a cancer tissue, or the tissue surrounding a cancer tissue. In one embodiment of said method, said agent is conjugated to the copolymer of the disclosure, such as conjugated via a bond or a linking group.

### Examples

### Example 1: Synthesis of Copolymer and Copolymer-Dye-Conjugates

### Materials and Methods

The copolymers were synthesised using either the RAFT Agent 2-cyano-2-propyl dodecyl trithiocarbonate (giving the AASTY_{12.5}) 2-copolymer or (Dodecylthiocarbonothioylthio)-2-methylpropionic acid 3-azido-1-propanol ester (giving the copolymer AASTY_{9.9}-N3), the initiator azobisisobutyrunitrile (AIBN), and the monomers acrylic acid (AA) and styrene (STY) (with an initial molar ratio of (45:55)(AA:STY)). Schlenk flasks were charged with the reagents, and oxygen was removed using via 4 freeze-pump-thaw cycles. The mixtures were heated to 60 or 70° C. for 9 to 12 hours, reaching 95% monomer conversion for AASTY_{12.5} and 69% for AASTY_{9.9}-N3. The products were solubilized in diethyl ether, precipitated into heptane, and dried in vacuo to yield yellow crisp solids. For AASTY_{12.5}, the polymer was solubilized in (water:ethanol) (1:3) with 30% H₂O₂ and incubated at 70° C. overnight to remove the dodecyl trithiocarbonate (ttc) end group, yielding a colorless solution. That polymer was precipitated into deionized water and collected by centrifugation. All the final polymer products were converted to partial sodium salts by solubilization in deionized water with the addition of NaOH (1 M) until the pH was at 7-7.5, and the opaque mixtures were filtered and lyophilized. The number average molecular weights (Mₙ), weight average molecular weights (M_{w}) and dispersities (D=Mₙ/M_{w}) were measured using a Dionex Ultimate 3000 system. Detection was performed by a Dawn Heleos II Multi Angle Light Scattering detector, and an Optilab rEX refractive index detector. Gell Permeation Chromatography (GPC) was done in a Superose 6 Increase column (10/300, Cytiva). Data were analyzed with Astra 7.0, using a dn/dc of 0.170 mL/g.

The polymer was dissolved in methanol at 50 mg/mL and equimolar Cyanine7-DBCO (NIR fluorescent dye with cycloalkyne moiety for the conjugation with azides—Lumiprobe GmbH) was added and the reaction left overnight at room temperature. The methanol was evaporated by nitrogen flow, and the solids were dissolved in water. The dye-conjugated polymer was purified by size exclusion column chromatography, and lyophilized to yield dark green solids.

### Results

All products were analysed by SEC-MALS coupled to a UV detector. The Cy7 dye absorbs the MALS laser, hindering mass determination, though the elution time of the polymer is identical to the non-conjugated polymer. The copolymers and conjugates produced are shown in Table 1.

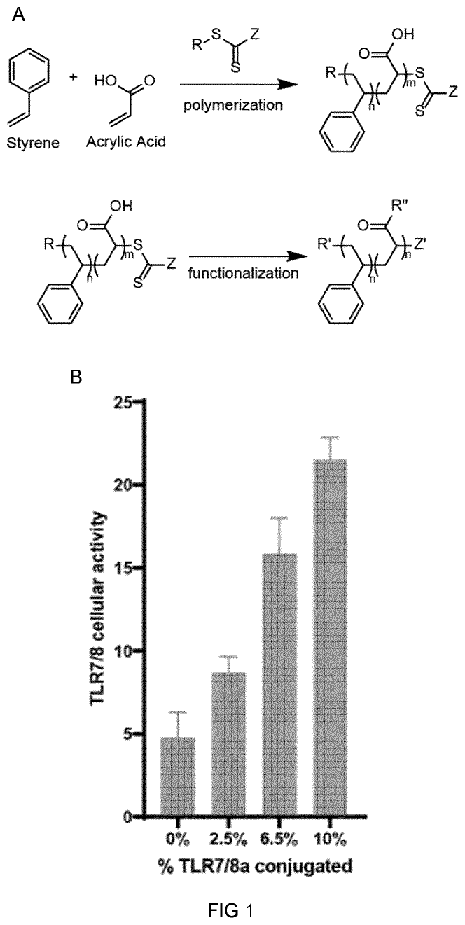

TABLE 1
summary of conjugates produced.

| | Carrier | Z′ group | R′ group | R″ group |
|---|---|---|---|---|
| Compound | polymer | (cf. FIG. 1A) | (cf. FIG. 1A) | (cf. FIG. 1A) |
| AASTY _{12.5} | | OH | CH₂(CH₃)₂CN | OH |
| AASTY _{12.5}- | AASTY _{12.5} | OH | CH₂(CH₃)₂CN | TLR7/8a |
| TLR7/8a | | | | |
| AASTY _{9.9}-N3 | | ttc-C₁₂H₂₅ | CH₂(CH₃)₂COO(CH₂₎₃N₃ | OH |
| AASTY _{9.9}- | AASTY _{9.9}-N3 | ttc-C₁₂H₂₅ | CH₂(CH₃)₂COO(CH₂)₃HN- | OH |
| Cy7 | | | Cy7 | |
| AASTY _{9.9}- | AASTY _{9.9}-N3 | ttc-C₁₂H₂₅ | CH₂(CH₃)₂COO(CH₂)₃N₃ | TLR7/8a |
| TLR7/8a | | | | |
| AASTY _{9.9}- | AASTY _{9.9}-N3 | ttc-C₁₂H₂₅ | CH₂(CH₃)₂COO(CH₂)₃HN- | TLR7/8a |
| TLR7/8a | | | Cy7 | |

### Conclusion

The azide RAFT agent was used to make AASTY polymers, and enabled ‘click’ conjugation to the R terminus of the RAFT agent.

### Example 2: Synthesis of Copolymer Conjugated to TLR Agonist

### Materials and Methods

The AASTY copolymers were added to 5, 10 or 15% molar equivalent to AA content of the TLR7/8 agonist 4-Amino-2-(ethoxymethyl)-1H-imidazo[4,5-c]quinoline-1-propanamine (referred to as “TLR7/8a”) in DMF. Most of the agonist was solubilized by gentle heating, and 2 molar equivalents (to the TLR7/8a) of NHS with 1 molar equivalent of N, N-Diisopropylcarbodiimide were added to the samples. The reactions were left at room temperature overnight, and the products were purified the next day by size exclusion chromatography, and lyophilized to yield white solids.

A fraction of the conjugates where solubilized in methanol with 1 molar equivalent of Cyanine7-DBCO. The reaction was left at room temperature overnight and purified by size exclusion chromatography, and lyophilized to yield dark green solids.

### Results

Characterization by ¹H NMR confirmed and quantified the conjugation of TLR7/8a via its aminopropyl group to the AA carboxylic acid of the copolymer.

### Example 3: Copolymer Constructs Reside in Tissues for Exceptional Durations In Vivo

### Materials and Methods

AASTY constructs with a fluorescent dye (AASTY-Cy7) were administered in healthy mice via different routes of administration. The biodistribution was monitored over the course of 2 weeks.

### Results

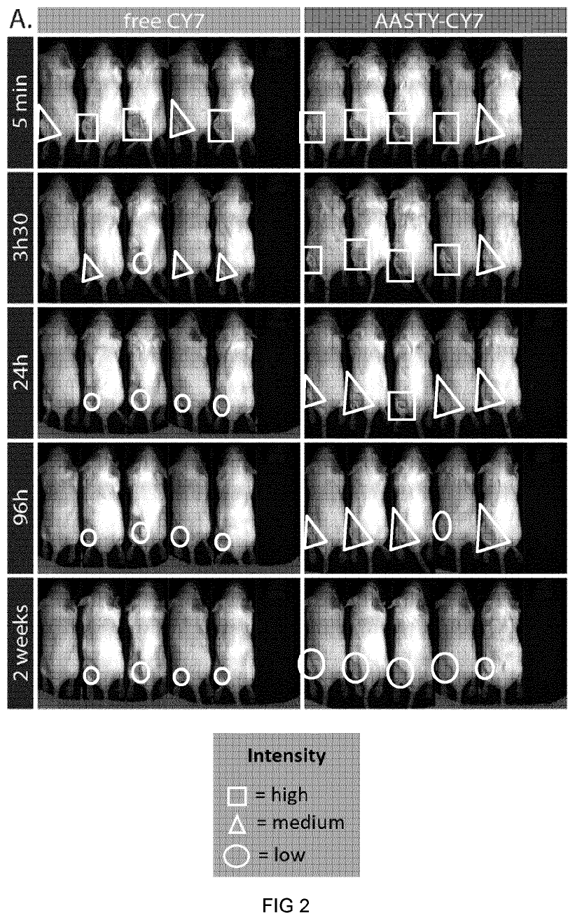

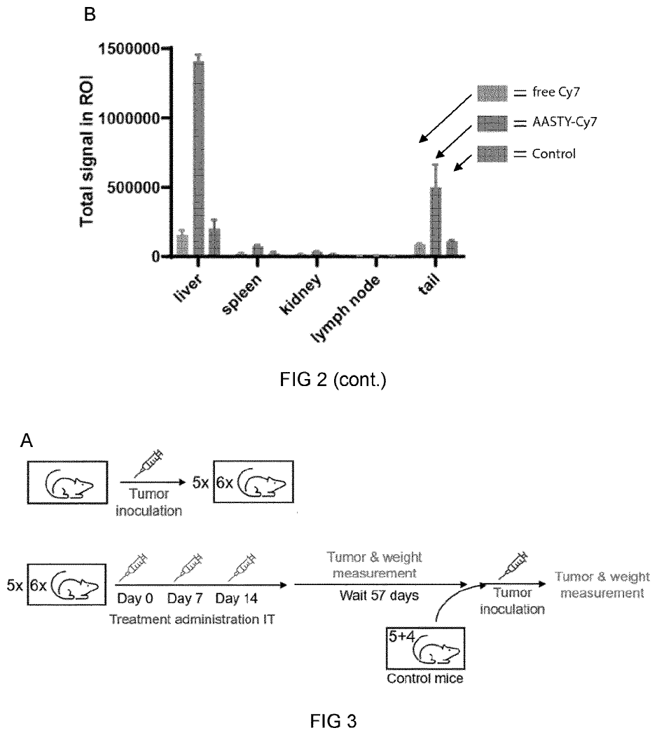

After subcutaneous (SC) injection of the AASTY_{9.9}-Cy7 of Example 1, most of the fluorescence signal remained in the near area of the initial injection depot and did not seem to significantly diffuse away after the formulation was absorbed by the SC tissue (FIG. 2A). While the integrated signal density slowly decreased over time, a large portion of the signal was still clearly visible after 2 weeks and with an unchanged distribution, unlike for the free dye control which was mostly cleared by renal excretion over the first few hours after administration. This suggests that the AASTY-Cy7 conjugate strongly and rapidly adheres to local tissues for extended periods of time. Image analysis estimated the compound's half-life to be 58 hours, which is remarkable for water soluble material. To exclude the possibility that the Cy7 dye was cleaved from the AASTY conjugates and misleading our interpretation, an equivalent dose of the DBCO-Cy7 dye in the form used for conjugation was solubilized in 5% DMSO-DPBS and injected SC in a similar manner. That control experiment showed that despite not being cleared through renal excretion, the free dye is rapidly metabolized at its injection site with most of its signal being gone after 1 week, which confirms that the conjugates exhibit different PK profiles than the free forms of the dye. Considering no significant signal was detected outside the injection region in vivo, the mice were autopsied at the end of the 2 weeks to extract organs and analyse the signal distribution with greater sensitivity. The isolated organ images revealed that signal was emanating from a spot on the inguinal lymph node on the injection side and residual signal was also visible in the liver. This suggests that the compound is slowly cleared via hepatic and lymphatic routes. No signs of toxicity or inflammation were observed during the study, suggesting that the copolymer itself (i.e. not conjugated to any drug) is neither significantly toxic nor immunogenic. After intravenous (IV) injection in the tail vein, the compound rapidly reached systemic circulation, as told by signal emanating from the paws, snout and eyes, for the first few hours after injection. Like for SC injection, an important part of the signal remained close to the injection point (the tail) and did not diffuse for the whole duration of the study after having settled. Here an important signal was visible in the liver from the first acquisition time until the end of the 2 weeks. Image analysis of the liver region revealed a smaller half-life for the compound, of about 17 hours, but the signal was still visible after 2 weeks. The organ extraction and analysis after autopsy (in which the tail of the mouse was included) confirmed important accumulation of the compound in the liver and also showed small yet significant accumulation in the spleen, which was not observed for SC administration (FIG. 2B). No signal was reported neither in the inguinal lymph node or the kidney though. Here again, no signs of toxicity or inflammation were to be reported.

Together these two studies suggest that AASTY has the ability to deeply adhere to the local tissues in which it is injected and stain them for remarkably long periods of time. Yet the data also suggests that some part of it is slowly cleared overtime through the hepatic and lymphatic route. The control experiment also points out that the dye is likely not cleaved from AASTY during its in vivo residency. Adding on top of that, the good tolerance of the mice to the compound and the apparent absence of immunogenicity, this makes AASTY conjugates an ideal candidate for tissue staining or local drug exposure.

### Example 4: Copolymer Conjugate Show Good Retention in Solid Tumours in a Murine Model

### Materials and Methods

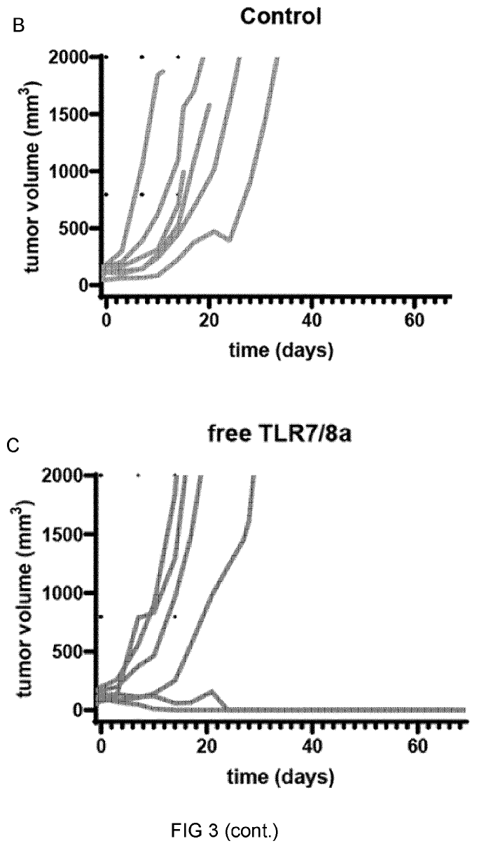

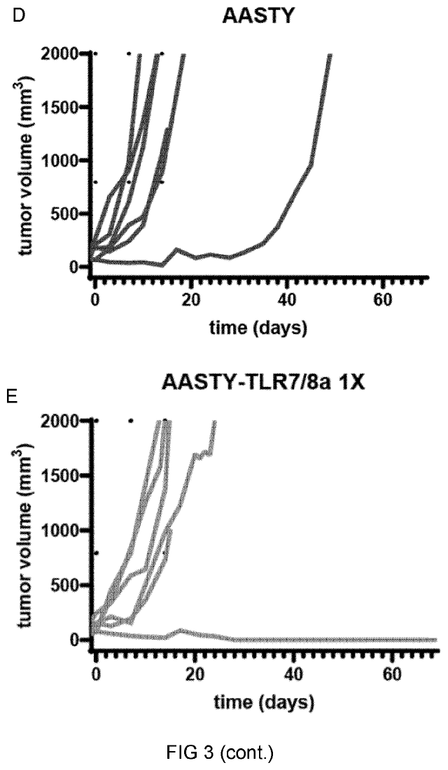

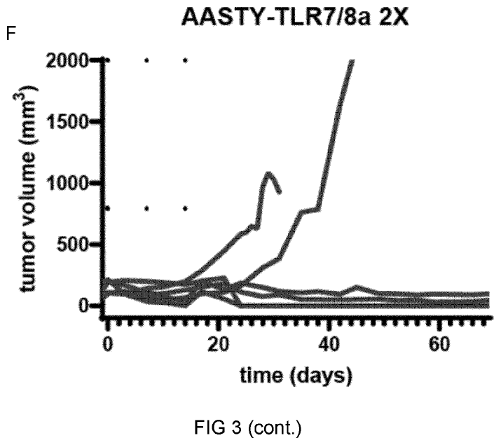

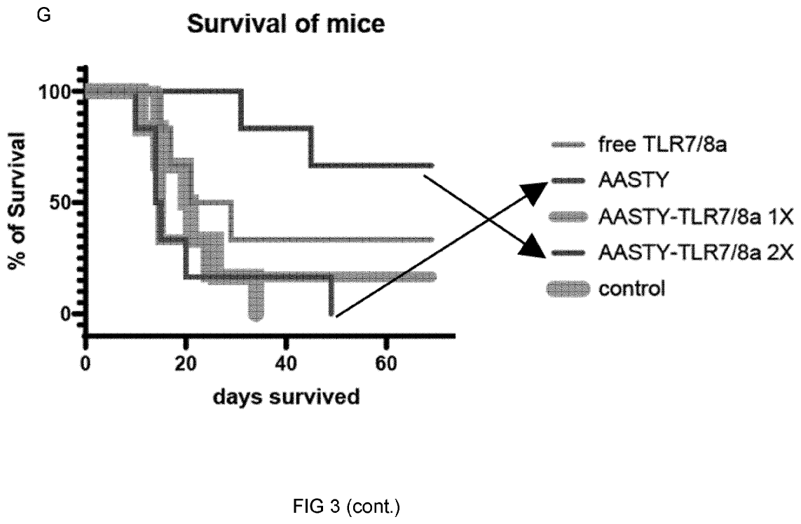

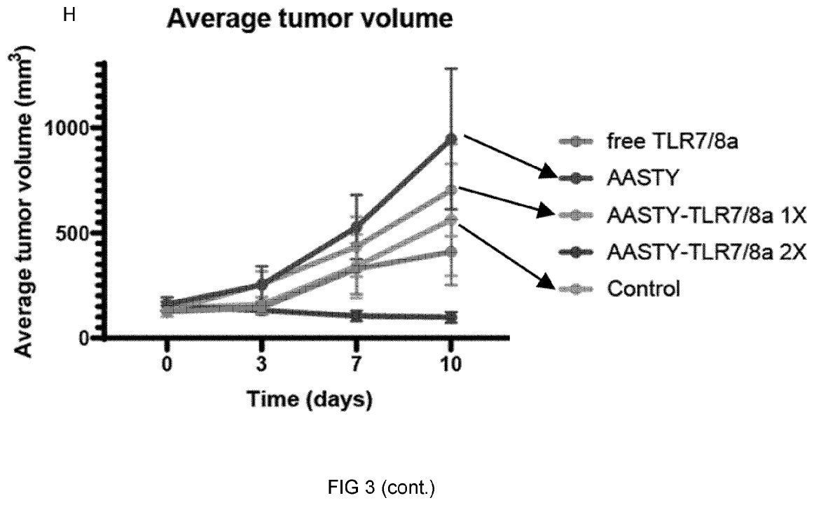

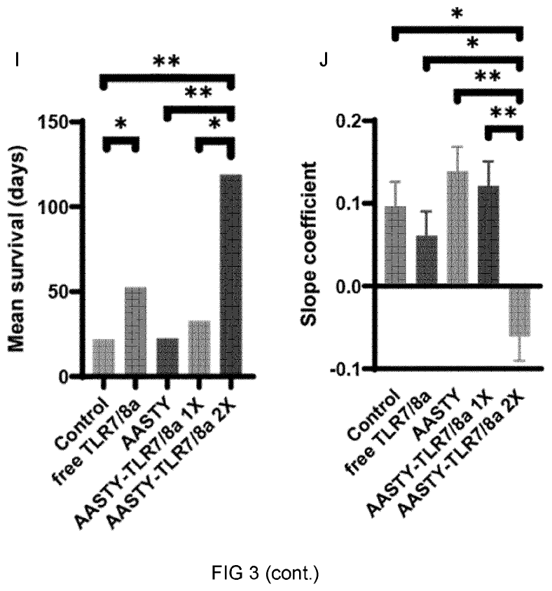

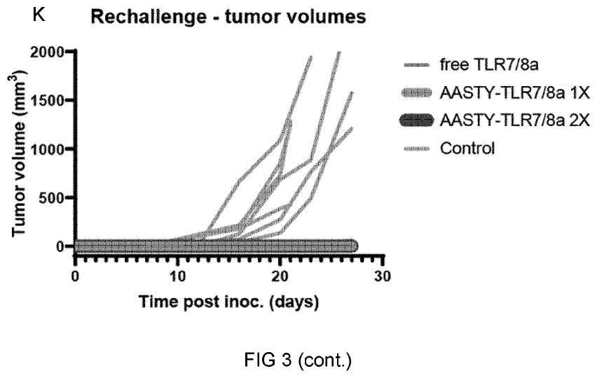

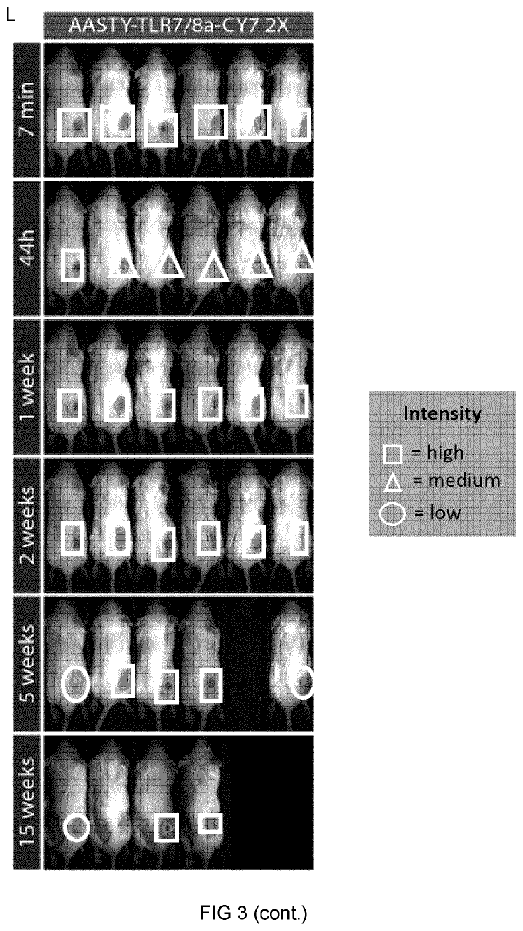

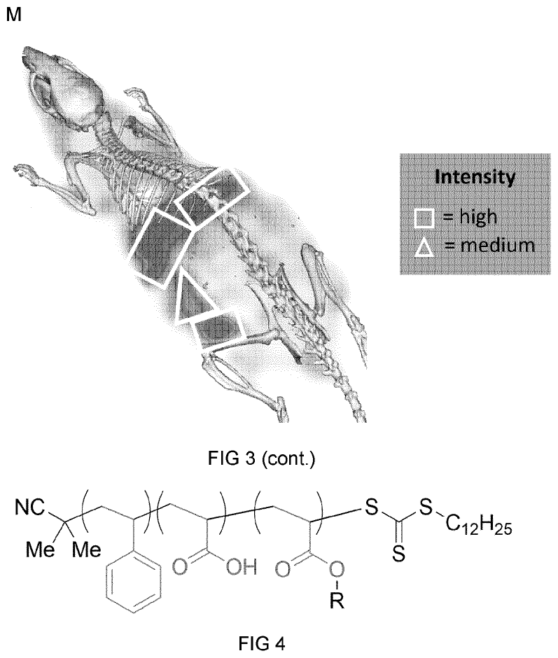

Healthy Balb/cJRj mice were injected with 100 μL of Murine colon carcinoma CT26 cancer cells (3×105) in RPMI media subcutaneously on the right flank. Mice bearing tumors with volumes between 50 and 200 mm³ were randomized and rearranged into new cages by blocking for initial tumor volume, and each cage received one of the four treatments described in Table 2 administered intratumorally (50 μL). A total of 3 injections, separated by 7 days each, were made for each treatment. Table 2 shows a summary of the tested treatments and FIG. 3A shows an overview of the study.

TABLE 2
Summary of tested treatments.

| | | | | TKR7/8a | Cy7 |
|---|---|---|---|---|---|
| Treatment | | Concen- | | dose | dose |
| name | Compound | tration | buffer | (mg/kg) | (nmol) |
| Free | TLR7/8a | 2.1 | 3.5% | 5 | 1.5 |
| TLR7/8a | | | DMSO- | | |
| | | | DPBS | | |
| AASTY | (AASTY _{9.9}:AASTY _{9.9}- | 18.4 | DPBS | 0 | 1.5 |
| | Cy7) (1:90) | | | | |
| AASTY- | (AASTY _{9.9}-TLR7/8a- | 9.2 | DPBS | 2.5 | 1.5 |
| TLR7/8a | Cy7:AASTY _{9.9}- | | | | |
| 1x | TLR7/8a) (1:45) | | | | |
| AASTY- | (AASTY _{9.9}-TLR7/8a- | 18.4 | DPBS | 5 | 1.5 |
| TLR7/8a | Cy7:AASTY _{9.9}- | | | | |
| 2x | TLR7/8a) (1:90) | | | | |

83 days after the first tumor inoculation, the surviving mice were re-challenged by a second inoculation of CT26 cancers cells (same dose) on the left flank of the mice along with a control group getting their first inoculation. The mice were monitored for weight and their tumors were measured using an electronic caliper twice a week, and fluorescence imaging was performed at regular intervals for all formulations containing Cy7 moieties. The autopsies were performed within a week after euthanasia (and stored at −20° C. in the meantime) and resected tumors and both inguinal lymph nodes were included. The average signal of each treatment group for each organ was corrected and aligned assuming a first order decay based on the half-life estimated with the in vivo scans, with consideration that all mice were euthanized at different moments of the study. The biodistribution of the compound was further assessed by injecting a single dose of AASTY-TLR7/8a 2× and (50 μL, intratumor) and scanning the mice by Fluorescence Tomography (MILabs U-CT in FLT mode). The scans were performed 5 to 6 days after euthanasia of the mice, which had been stored at −20° C. between euthanasia and scanning. The reconstructed images were analyzed and 3D-rendered using Imalytics Preclinical 2.1 During the study, tumor volumes were calculated as (length×width)²/2 and the mice were euthanized when tumor volumes reached 2000 mm³. Humane endpoints included general signs of misthriving mice, significant weight loss (15% of initial weight or 10% overnight weight loss), or presence of tumor wounds larger than 8 mm.

### Results

Intratumor administration data showed an extremely long residence time for the AASTY conjugates, with a half-life estimated at 41 days and most of the signal still clearly visible when the study was terminated after 117 days (acquisition week 15) (FIG. 2L). A large amount of the compound was retained in the tumor (where it were injected) and distributed in most of its volume. For the mice that were cured, the signal remained in the area neighboring the tumor scar, suggesting that the compound was redistributed in the surrounding tissues as the tumor cells were eliminated. Like after IV-administration, the corrected data from the autopsies revealed that a large amount of the signal was also observed in the liver, and a small amount of the AASTY-TLR7/8a conjugates also reached the spleen. While still nothing was detected in the kidneys and the left lymph node (opposite flank to the tumor), a significant signal was detected for the AASTY formulations in the right lymph node (same flank as the tumor), with an extensively higher amount for the high AASTY-TLR7/8a dose.

### Conclusion

Conjugates of AASTY show high residence time after intratumoral injection, exceeding the half-life of both SC and IV injections. The low biodistribution to the kidneys indicates that the disclosed conjugates may few kidney related adverse effects than other chemotherapies, which can damage the kidneys, the ureters, and the bladder.

### Example 5: TLR7/8a-Copolymer Conjugate Reduces Solid Tumour Volume in Murine Model and Improves Survival Rate

### Materials and Methods

Materials and methods were as outlined for Example 3.

### Results

The monitoring of tumor kinetics showed that most tumors grew exponentially rapidly after the beginning of the study for every mice groups (FIG. 2B to 2E) except for the high AASTY-TLR7/8a dose treatment group (FIG. 2F). Here, the influence of two parameters was assessed: the dose of the TLR7/8a (with a non-lethal high dose in the range expected to trigger an immune response and a half-dose below that range), and the dose of AASTY (kept at a constant ratio to the TLR7/8a). First, the results of the AASTY and AASTY-TLR7/8a 1× treatment groups suggest that at both doses, AASTY itself does not feature antitumor activity. TLR7/8a alone injected at a high dose intratumorally shown a potential minor improvement in tumour treatment compared to the control, considering that two out of six mice recovered whereas the remaining four mice did not respond to the treatment. On interpretation of these results is the effect of the extremely high potency of these compounds in contrast to their rapid diffusion out of the tumour microenvironment after injection. The copolymer-drug-conjugate of the disclosure set out to provide sustained local residence. For AASTY-TLR7/8a 1×, no significant improvement was observed. Conversely, for AASTY-TLR7/8a 2×, a total absence of tumour growth was seen in the first 20 days, and an overall negative tumor growth rate in the first 10 days (FIGS. 2F, 2H, and 2J). The overall higher survival rate is in overall higher, compared with the low dose treatment, with only two out of six mice growing tumours after 20 days; the remaining four mice completely recovered (FIGS. 2G and 2I).

### Conclusion

TLR7/8a-copolymer conjugate reduces solid tumour volume in murine model and improves survival rate at the appropriate dose.

### Example 6: TLR7/8a-Copolymer Conjugate Prevents Tumour Growth in Tumour Inoculation Study in Murine Model

### Materials and Methods

83 days after the first tumour inoculation, the surviving mice were re-challenged by a second inoculation of CT26 cancers cells (same dose) on the left flank of the mice along with a control group getting their first inoculation. The mice were monitored for weight and their tumours were measured using an electronic caliper twice a week, and fluorescence imaging was performed at regular intervals for all formulations containing Cy7 moieties.

### Results

The surviving mice were re-challenged with tumour cells 83 days after the first inoculation and no tumour growth was observed (FIG. 2K).

### Conclusion

Mice surviving the study had an adaptive response, and are resistant to CT26.

### Example 7: Copolymer is Retained in Injected Tissue; Small Amounts Distribute Slowly Through the Lymphatic System in Murine Model

### Materials and Methods

To get a better insight in the biodistribution dynamics after intratumor administration, fluorescence tomography scans where performed 5 to 6 days after the administration of a single dose of AASTY-TLR7/8a 2× on a small group of mice.

### Results

The compound diffused through all the bean-shaped tumor with a signal maximum at the injection point and mostly accumulated in the liver (FIG. 2M). The new information brought by this study is that the compound apparently distributed in the entire lymphatic system, as signal can be detected in a globally symmetric network of canals connected by higher intensity nodes that more or less align with known locations of lymph nodes (notably, among others, the mandibular, cervical, axillary, brachial, lumbar and codal).

### Conclusion

From this and other examples disclosed here, the possible following mechanism of biodistribution action is likely: after injection, copolymer/copolymer conjugates will rapidly adhere to local tissues, likely by exploiting its amphiphilicity of the copolymer to incorporate itself in lipid membranes or even inside cells. If it is injected IV or in a highly vascularized tissue, a fraction of it will flow in the systemic circulation and it will rapidly accumulate mainly in the liver and, to a lesser extend, to the spleen.

### Example 8: The Copolymer MAASTY Exhibits a Similar Biodistribution Profile as AASTY

### Materials and Methods

Poly(methacrylic acid-co-styrene) (MAASTY) was synthesized similarly as described in Example 1, using the RAFT Agent 2-cyano-2-propyl dodecyl trithiocarbonate 2-(Dodecylthiocarbonothioylthio)-2-methylpropionic acid 3-azido-1-propanol ester, the initiator azobisisobutyronitrile (AIBN), and the monomers methacrylic acid (MAA) and styrene (STY) (with an initial molar ratio of (50:50) (MAA: STY)). As an alternative method to using Schlenk flasks, here a glass ampoule was charged with the reagents, oxygen was removed via 4 freeze-pump-thaw cycles, and the ampoule was sealed under vacuum with a torch ligher. The mixture was heated to 55° C. for 16 hours, reaching an estimated monomer conversion of 85% (estimated based on the viscosity). The product was solubilized in diethyl ether, precipitated into heptane, and dried in vacuo to yield a yellow crisp solid.

The copolymer was then dissolved in methanol at 25 mg/mL and equimolar Cyanine7-DBCO (same as in Example 1) was added and the reaction left overnight at room temperature. The methanol was evaporated by nitrogen flow, and the solids were dissolved in water. The dye-conjugated polymer was purified by size exclusion column chromatography, and lyophilized to yield a dark green solid.

As a reference, a new batch of AASTY-Cy7 was made following a similar protocol as in Example 1 with the RAFT agent 2-(dodecylthiocarbonothioylthio)-2-methylpropionic acid 3-azido-1-propanol ester, (45:55) (AA: STY), but using sealed ampoules instead of Schlenk flasks as for MAASTY. The mixture was heated to 60° C. for hours, reaching a conversion of 85% (measured by ¹H-NMR) and giving the copolymer AASTY_{7.1} (Mₙ=7.1 kDa, estimated by ¹H-NMR only). As for MAASTY, the copolymer was then conjugated to Cy7 at 25 mg/mL copolymer to yield AASTY_{7.1}-Cy7.

The two Cy7 copolymer constructs were administered in mice via subcutaneous administration. All mice were bearing palpable CT26 tumors but were injected in the flank outside the direct vicinity of the tumors. The biodistribution was monitored via Fluorescence Tomography over the course of 5 days.

### Results

The biodistribution of the AASTY_{7.1}-Cy7 construct (FIG. 5) was consistent with the prior biodistribution data from Example 3 and 7 (note that in Example 7, the copolymer was administered intratumorally and not subcutaneously, which explains the stronger liver accumulation compared to that new dataset). Most of the fluorescence signal remained in the near area of the initial injection depot and a small fraction spread to the lymphatic network over time, with a majority of the signal still measurable 5 days after injection.

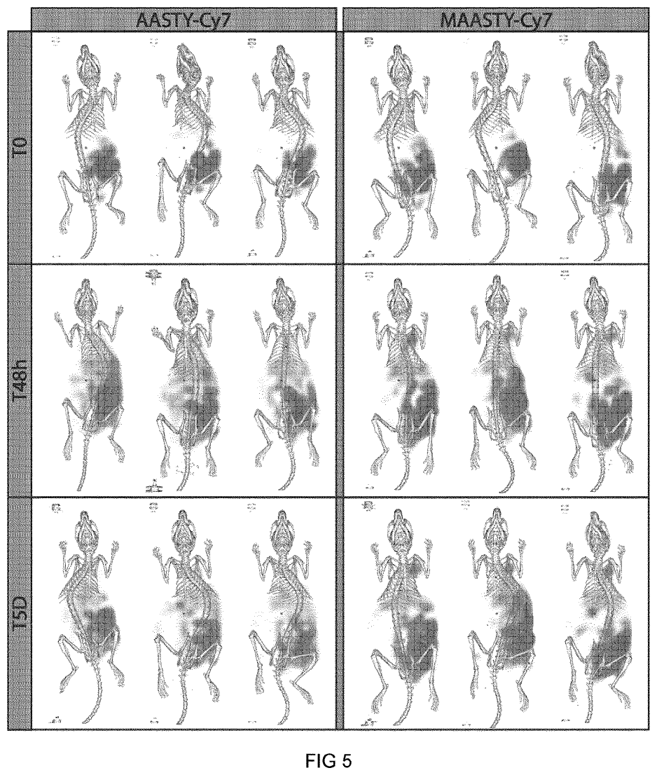

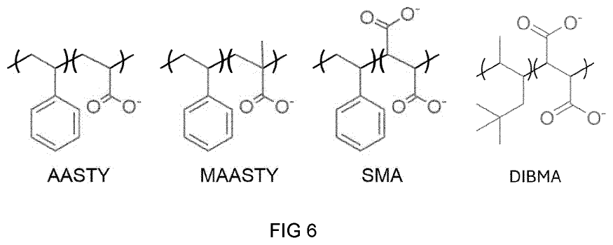

The MAASTY-Cy7 conjugate seemed to distribute following the same pattern, with a slightly higher signal remaining after 5 days but very well mimicking the results of AASTY-Cy7 in terms of tissue residence and biodistribution. These results demonstrate that both polymers share a common pharmacokinetic profile, which is attributed to their structural and physicochemical similarity (FIG. 6) and which indicate that they exhibit very similar type of interaction with biological tissues. For this reason, the copolymers SMA and DIBMA are also envisioned as being useful for retaining cargos in tissues. It is to be noted that the mice in this biodistribution study were bearing tumors but based on the consistency of AASTY-Cy7 with the results from healthy mice in Example 3 and the similarity of the biodistribution profile for both AASTY and MAASTY in FIG. 5, this assumption can reasonably be extrapolated to healthy mice.

### Conclusion

Due to their structural similarity, AASTY and MAASTY exhibited similar pharmacokinetic profiles, both showing exceptional tissue residence properties. It is expected that both copolymers will perform to a similar level at cancer treatment when conjugated to a TLR7/8 agonist as in Example 5 and 6. Based on these findings, it is also envisioned that other polymers like poly(styrene-co-maleic acid) (SMA) and poly(diisobutylene-co-maleic acid) (DIBMA) would behave similarly in vivo and exhibit the same type of pharmacokinetic profiles regarding tissue residence, because they like AASTY and MAASTY are amphiphilic copolymers, comprising both hydrophobic and hydrophilic subunits (see FIG. 6).

### REFERENCES

Slezak et al., Tumor Cell-Surface Binding of Immune Stimulating Polymeric Glyco-Adjuvant via Cysteine-Reactive Pyridyl Disulfide Promotes Antitumor Immunity, ACS Cent. Sci. 2022, 8, 1435-1446.
Shukla et al., Structure-Activity Relationships in Human Toll-like Receptor 7-Active Imidazoquinoline Analogues, J Med Chem. 2010 Jun. 10; 53 (11): 4450-4465. WO 2022/226032 A1.

1. A copolymer-drug-conjugate comprising an amphiphilic copolymer and 3 or more anti-cancer drug moieties conjugated to said amphiphilic copolymer.

2. The copolymer-drug-conjugate according to claim 1, wherein the amphiphilic copolymer is an anionic amphiphilic copolymer.

3. The copolymer-drug-conjugate according to any one of the preceding claims, wherein the amphiphilic copolymer is polymerized from:
a. styrene and/or diisobutylene, and
b. acrylic acid, methacrylic acid, and/or maleic acid.

4. The copolymer-drug-conjugate according to any one of the preceding claims, wherein the amphiphilic copolymer is a poly(acrylic acid-co-styrene) copolymer, a poly(methacrylic acid-co-styrene) copolymer, a poly(maleic acid-co-styrene) copolymer, or a poly(diisobutylene-co-maleic acid) copolymer.

5. The copolymer-drug-conjugate according to any one of the preceding claims, wherein the anti-cancer drug moieties are conjugated to said amphiphilic copolymer via a bond or a linking group.

6. A copolymer-drug-conjugate having the structure of formula (III): 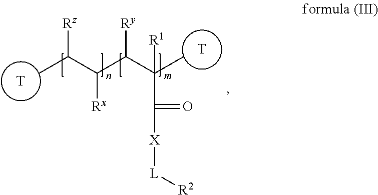
wherein:
each R¹ is independently selected from CH₃ and H,
each R^{y} is independently selected from H, COOH, and COX-L-R²,
each R^{z} is independently selected from H and CH₃,
each R^{x} is independently selected from 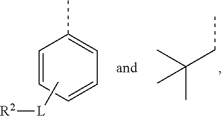
each X is independently selected from O, S, or NH,
each L is independently a bond or a linking group,
each R² is independently selected from H or an anti-cancer drug, wherein at least 3 of R² are an anti-cancer drug moiety,
each T is the same or different terminal groups,
n is between 5 and 500,
m is between 5 and 500,
wherein the copolymer is a random, alternating, or statistical copolymer.

7. The copolymer-drug-conjugate according to any one of the preceding claims, wherein the copolymer-drug-conjugate has the structure of formula (I): 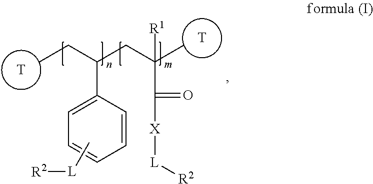
wherein:
each R¹ is independently selected from CH₃ and H,
each X is independently selected from O, S, or NH,
each L is independently a bond or a linking group,
each R² is independently selected from H or an anti-cancer drug, wherein at least 3 of R² are an anti-cancer drug moiety,
each T is the same or different terminal groups,
n is between 5 and 500,
m is between 5 and 500,
wherein the copolymer is a random, alternating, or statistical copolymer.

8. The copolymer-drug-conjugate according to any one of the preceding claims, wherein R¹ is CH₃.

9. The copolymer-drug-conjugate according to any one of the preceding claims, wherein the copolymer set out in formula (I) is a poly(methacrylic acid-co-styrene) copolymer or a poly(acrylic acid-co-styrene) copolymer.

10. The copolymer-drug-conjugate according to any one of the preceding claims, wherein the copolymer-drug-conjugate has the structure of formula (II):

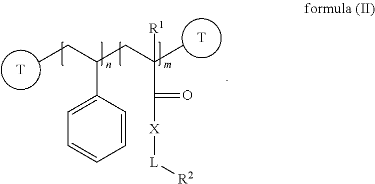

11. The copolymer-drug-conjugate according to any one of the preceding claims, wherein X is selected from O, S, or NH, such as wherein X is O.

12. The copolymer-drug-conjugate according to any one of the preceding claims, wherein L is a bond.

13. The copolymer-drug-conjugate according to any one of the preceding claims, wherein L is a linking group selected from PEG, alkylenes, triazoles, and a group form from reaction between a haloalkyl and a nucleophile.

14. The copolymer-drug-conjugate according to any one of the preceding claims, wherein the copolymer of the copolymer-drug-conjugate has a molecular mass of less than 20 kDa, such as less than 15 kDa, such as less than 14 kDa, such as less than 13 kDa, and/or wherein the copolymer has a molecular mass of at least 1 kDa, such as at least 3 kDa, such as at least 5 kDa, such as at least 7 kDa, such as at least 9 kDa.

15. The copolymer-drug-conjugate according to any one of the preceding claims, wherein the anti-cancer drug is an anti-tumour drug.

16. The copolymer-drug-conjugate according to any one of the preceding claims, wherein the anti-tumour drug is an anti-tumour drug with effect against solid tumours.

17. The copolymer-drug-conjugate according to any one of the preceding claims, wherein the anti-cancer drug is a Toll-Like Receptor (TLR) agonist, such as a TLR7 agonist, a TLR8 agonist, or a TLR7/8 agonist.

18. The copolymer-drug-conjugate according to any one of the preceding claims, wherein the anti-cancer drug has the structure of formula (A-I), formula (A-II), formula (A-III), or formula (A-IV): 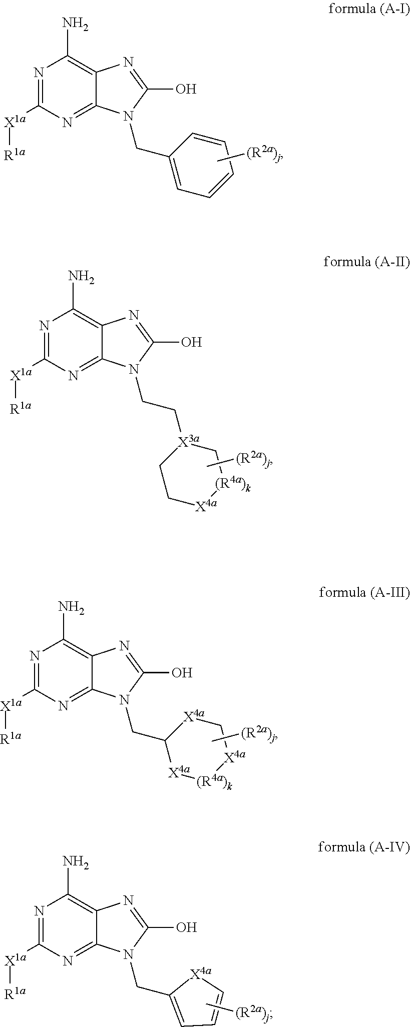
wherein
X^{1a} is —O—, —S—, or —NR^{Ca};
R^{1a} is hydrogen, (C₁₋₁₀)alkyl, substituted (C₁₋₁₀)alkyl, C₆₋₁₀aryl, or substituted C₆₋₁₀aryl, C₅₋₉heterocyclic, substituted C₅₋₉heterocyclic;
R^{Ca} is hydrogen, (C₁₋₁₀)alkyl, or substituted C₁₋₁₀alkyl; or R^{Ca} and R^{1a} taken together with the nitrogen atom to which they are attached form a heterocyclic ring or a substituted heterocyclic ring;
each R^{2a} is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted-C(O)—(C₁-C₆)alkyl, —C(O)—(C₆-C₁₀) aryl (aroyl), substituted-C(O)—(C₆-C₁₀) aryl, —C(O)OH (carboxyl), —C(O)O(C₁-C₆)alkyl (alkoxycarbonyl), substituted-C(O)O(C₁-C₆)alkyl, —NR^{aa}R^{ba}, —C(O) NR^{aa}R^{ba} (carbamoyl), halo, nitro, or cyano, or R^{2a} is absent;
each R^{aa} and R^{ba} is independently hydrogen, (C₁-C₆)alkyl, substituted (C₁-C₆)alkyl, (C₃-C₅) cycloalkyl, substituted (C₃-C₈) cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;
wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxy C₁₋₆alkylene, amino, cyano, halo, or aryl;
j is 0, 1, 2, 3 or 4;
X^{3a} is —N— or —CH—;
R^{4a} is —CH₂— or —CH(R^{2a})—;
k is 0 or 1;
X^{4a} is —O—, —S—, —NH—, —N(R^{da})—, —CH₂—, or —CH(R^{2a})—;
each R^{da} is independently —OH, (C₁-C₆)alkyl, substituted (C₁-C₅)alkyl, (C₁-C₆)alkoxy, substituted (C₁-C₆)alkoxy, —C(O)—(C₁-C₆)alkyl (alkanoyl), substituted-C(O)—(C₁-C₆)alkyl, —C(O)—(C₆-C₁₀) aryl (aroyl), substituted-C(O)—(C₆-C₁₀) aryl, —C(O)O(C₁-C₆)alkyl (alkoxycarbonyl), substituted-C(O)O(C₁-C₆)alkyl, —C(O) NR^{aa}R^{ba} (carbamoyl);
or a tautomer thereof.

19. The copolymer-drug-conjugate according to any one of the preceding claims, wherein the anti-cancer drug has the structure of formula (B-I): 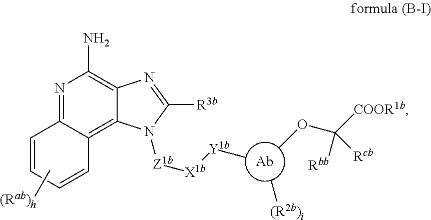
wherein
R^{1b} represents (C₁₋₈)alkyl, (C₃₋₈) cycloalkyl, or a 3- to 8-membered saturated heterocyclic ring group comprising a O atom, wherein R^{1b} is optionally substituted by one or more substituents independently selected from halogen, cyano, hydroxyl and (C₁₋₃)alkoxy;
Z^{1b} represents a (C₂₋₆)alkylene group, wherein a carbon atom in Z^{1b} which is not adjacent to a nitrogen atom may be replaced with an oxygen atom;
X^{1b} represents NR^{5b}, >N—COR^{5b}, >N—CONR^{5b}R^{5ab}, CONR^{5b}, NR^{5b}CO, NR^{5b}CONR^{6b} or NR^{6b}CONR^{5b};
Y^{1b} represents a single bond or (C₁₋₆)alkylene;
each R^{2b} is independently selected from halogen, cyano, hydroxy, thiol, (C₁₋₃)alkyl, (C₁₋₃) hydroxyalkyl, (C₁₋₃)haloalkyl, (C₁₋₃)alkoxy, (C₁₋₃)haloalkoxy, (C₁₋₃)alkylthio, (C₁₋₃)alkylsulfonyl and (C₁₋₃)alkylsulfinyl;
R^{3b} represents C₁₋₆ alkyl optionally substituted by (C₁₋₆)alkoxy;
each R^{ab} is independently selected from halogen, cyano, hydroxy, thiol, (C₁₋₃)alkyl, (C₁₋₃) hydroxyalkyl, (C₁₋₃)haloalkyl, (C₁₋₃)alkoxy, (C₁₋₃)haloalkoxy, (C₁₋₃)alkylthio, (C₁₋₃)alkylsulfonyl and (C₁₋₃)alkylsulfinyl;
R^{5b} and R^{5ab} each independently represents hydrogen, a 3- to 8-membered saturated heterocyclic ring comprising a ring group O, S(O)ₚ or NR^{10b}, a (C₁₋₆)alkyl group or (C₃₋₆) cycloalkyl group, the latter two groups being optionally substituted by one or more substituents independently selected from NR^{7b}R^{8b} or R^{9b}, R^{7b} and R^{8b} each independently represent hydrogen, a 3- to 8-membered saturated heterocyclic ring comprising a ring group O, S(O)ₚ or NR^{10ab}, (C₁₋₆)alkyl or (C₃₋₆) cycloalkyl, the latter two groups being optionally substituted by one or more groups independently selected from halogen, cyano, S(O)_{q}R^{11b}, OR^{12b}, CO₂R^{12b}, OC(O)R^{12b}, SO₂NR^{12b}R^{13b} CONR^{12b}R^{13b}, NR^{12b}R^{13b}, NR^{12b}SO₂R^{14b}, NR^{12b}COR^{13b}, or a 3- to 8-membered saturated heterocyclic ring comprising a ring group O, S(O)ₚ or NR^{10bb},
or R^{7b} and R^{8b} together with the nitrogen atom to which they are attached form a 3- to 8-membered saturated heterocyclic ring comprising a ring nitrogen atom and optionally one or more further heteroatoms independently selected from nitrogen, oxygen, sulphur and sulphonyl, the heterocyclic ring being optionally substituted by one or more substituents independently selected from halogen, cyano, S(O)_{q}R^{15b}, OR^{15b}, CO₂R^{15b}, COR^{15b}, OC(O) R^{15b}, SO₂NR^{15b}R^{16b}, CONR^{15b}R^{16b}, NR^{15b}R^{16b}, NR^{15b}SO₂R^{17b}, NR^{15b}COR^{16b}, NR^{15b}CO₂R^{16b}, heteroaryl, (C₁₋₆)haloalkyl, (C₃₋₈) cycloalkyl and (C₁₋₆)alkyl, the latter two groups being optionally substituted by one or more groups independently selected from cyano, S(O)_{q}R^{18b}, OR^{18b}, CO₂R^{18b}, SO₂NR^{18b}R^{19b}, CONR^{18b}R^{19b} or NR^{18b}R^{19b};
R^{Ob} represents halogen, cyano, CO₂R^{20b}, S(O)_{q}R^{20b}, OR^{20b}, SO₂NR^{20b}R^{22b}, CONR^{20b}R^{22b}, NR^{20b}SO₂R^{21b}, NR^{20b}CO₂R^{21b}, NR^{20b}COR^{22b} or a 3- to 8-membered saturated heterocyclic ring comprising a ring group NR^{10cb};
R^{10b}, R^{10ab}, R^{10bb} and R^{10cb} independently represent hydrogen, CO₂R^{23b}, S(O)_{q}R^{23b}, COR^{24b}, or a (C₁₋₆)alkyl, (C₂₋₆)alkenyl, (C₂₋₆)alkynyl or (C₃₋₈) cycloalkyl group, each of which may be optionally substituted by one or more substituents independently selected from halogen, cyano, OR^{25b} or NR^{25b}R^{26b};
R^{6b}, R^{11b}, R^{12b}, R^{13b}, R^{15b}, R^{16b}, R^{18b}, R^{19b}, R^{20b}, R^{22b}, R^{24b}, R^{25b} and R^{26b} each independently represent hydrogen, (C₁₋₆)alkyl or (C₃₋₆) cycloalkyl;
R^{14b}, R^{17b}, R^{21b} and R^{23b} each independently represent (C₁₋₆)alkyl or (C₃₋₆)cycloalkyl;
h, i, p and q each independently represent an integer 0, 1 or 2; and
Ab represents a monocyclic or bicyclic (C₆₋₁₀) aryl or a monocyclic or bicyclic (C₅₋₁₂) heteroaryl group containing 1-3 heteroatoms; and
R^{bb} and R^{cb} independently represent hydrogen or (C₁₋₆)alkyl, or R^{bb} and R^{cb} combine together to form (C₃₋₈) cycloalkyl.

20. The copolymer-drug-conjugate according to any one of the preceding claims, wherein the anti-cancer drug has the structure of formula (C-I): 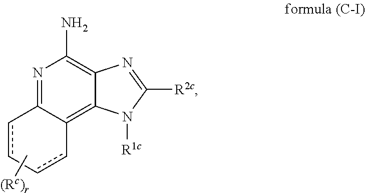
wherein
R^{1c} is selected from the group consisting of —(C₂₋₆)alkyl-N(R^{3c})₂, —(C₂₋₆)alkyl-NR^{3c}—SO₂—X^{c}—R^{4c}, and —(C₂₋₆)alkyl-NR^{6c}—SO₂—R^{7c};
X^{c} is a bond or —NR^{5c}—;
R^{4c} is alkyl, aryl, or heteroaryl;
R^{2c} is selected from the group consisting of hydrogen, alkyl, alkenyl, aryl, substituted aryl, heteroaryl, substituted heteroaryl, alkyl-O-aryl, alkyl-O-alkyl, alkyl-O-alkenyl, and alkyl or alkenyl substituted by one or more substituents selected from the group consisting of: OH, halogen, —N(R^{3c})₂, —CO—N(R^{3c})₂, —CO—(C₁₋₁₀)alkyl, —CO—O—(C₁₋₁₀)alkyl, —N₃, aryl, substituted aryl, heteroaryl, substituted heteroaryl, heterocyclyl, substituted heterocyclyl, —CO-aryl, —CO-(substituted aryl), —CO-heteroaryl, and —CO-(substituted heteroaryl);
each R^{3c} is independently selected from the group consisting of hydrogen and (C₁₋₁₀)alkyl,
R^{5c} is selected from the group consisting of hydrogen and (C₁₋₁₀)alkyl, or R^{4c} and R^{5c} can combine to form a 3 to 7 membered heterocyclic or substituted heterocyclic ring;
R^{6c} is selected from the group consisting of hydrogen and (C₁₋₁₀)alkyl;
R^{7c} is selected from the group consisting of hydrogen and (C₁₋₁₀)alkyl, or R^{5c} and R^{7c} can combine to form a 3 to 7 membered heterocyclic or substituted heterocyclic ring;
r is 0 to 4 and each R^{c} present is independently selected from the group consisting of (C₁₋₁₀)alkyl, (C₁₋₁₀)alkoxy, halogen and trifluoromethyl.

21. The copolymer-drug-conjugate according to any one of the preceding claims, wherein the TLR7, the TLR8, or the TLR7/8 agonist is selected from the list consisting of: Imiquimod, Resiquimod, Gardiquimod, 852A, Loxoribine, Bropirimine, 3M-011 (CAS no. 642473-62-9), 3M-052 (CAS no. 1359993-59-1), DSR-6434 (CAS no. 1059070-10-8), DSR-29133, SZU-101, SM-360320 (CAS no. 226907-52-4), SM-276001 (CAS no. 473930-22-2), VTX-2337 (CAS no. 926927-61-9), and 1-(3-aminopropyl)-2-(ethoxymethyl) imidazo[4,5-c]quinolin-4-amine.

22. The copolymer-drug-conjugate according to any one of the preceding claims, wherein the anti-cancer drug is a Stimulator of Interferon Genes (STING) agonist.

23. The copolymer-drug-conjugate according to any one of the preceding claims, wherein the anti-cancer drug is IL-2, IL-12, or IL-15, or a fragment thereof.

24. The copolymer-drug-conjugate according to any one of the preceding claims, wherein the anti-cancer drug is a radiopharmaceutical drug or a radioactive agent.

25. The copolymer-drug-conjugate according to any one of the preceding claims, wherein the copolymer-drug-conjugate comprises one type of anti-cancer drug.

26. The copolymer-drug-conjugate according to any one of the preceding claims, wherein the copolymer-drug-conjugate comprises two or more different anti-cancer drugs.

27. The copolymer according to any one of the preceding claims, wherein L is a linking group and wherein the moiety R² is conjugated to L at a carbon atom or a heteroatom such as N, O, or S in R².

28. The copolymer according to any one of the preceding claims, wherein L is a bond and wherein the moiety R² is conjugated to X at a carbon atom or a heteroatom such as N, O, or S in R².

29. The copolymer-drug-conjugate according to any one of the preceding claims, wherein T is independently selected from the group consisting of H, alkyl, substituted alkyl, nitrile, hydroxy, carboxyl, halogen, thiol, substituted thiol, acyl, substituted acyl, a group of formula X-I, a group of formula Y-I, a group of formula Y-II, a fluorescent dye, a diagnostic agent;
wherein the group of formula X-I has the structure: 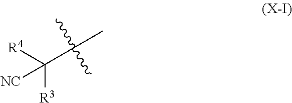
wherein R³ and R⁴ are each independently selected from hydrogen, alkyl, and substituted alkyl;
wherein the group of formula Y-I has the structure: 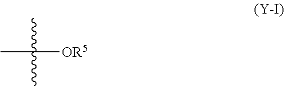
wherein R⁵ is selected from hydrogen, alkyl, and substituted alkyl; and
wherein the group of formula Y-II has the structure: 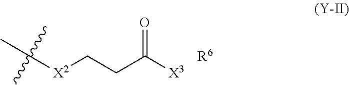
wherein R⁵ is selected from hydrogen, alkyl, and substituted alkyl, and
X² and X³ are each independently selected from S and O.

30. The copolymer-drug-conjugate according to any one of the preceding claims, wherein the fluorescent dye is selected from the group consisting of a cyanine dye such as indocyanine green or cyanine 7, a triarylmethane dye such as fluorescein, a thiazine dye such as methylene blue, LUM015, VGT-309, AVB-620, C-Dots, BLZ-100.

31. A pharmaceutical composition comprising the copolymer-drug-conjugate according to any one of the preceding claims.

32. The pharmaceutical composition according to claim 31, wherein the pharmaceutical composition is formulated for administration by injection, such as intratumoral injection.

33. A method for synthesising the copolymer-drug-conjugate according to any one of the preceding claims, said method comprising conjugating an anti-cancer drug to a precursor copolymer, such as a precursor poly(methacrylic acid-co-styrene) copolymer, a precursor poly(acrylic acid-co-styrene) copolymer, a precursor poly(maleic acid-co-styrene) copolymer, or a precursor poly(diisobutylene-co-maleic acid) copolymer.

34. The method according to claim 33, wherein the precursor copolymer has the structure of formula (III-P): 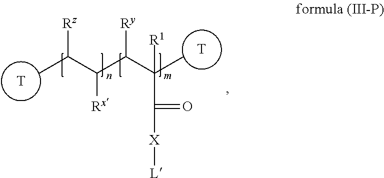
wherein R^{x′} is 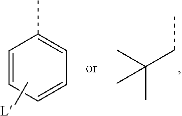
wherein each L′ is independently selected from H and a reactive handle, and
T, n, m, R¹, X, R^{z}, and R^{y} are as set out in any one of claims 6 to 11 or 27 to 28.

35. The method according to any one of claims 33 and 34, wherein the precursor poly(methacrylic acid-co-styrene) copolymer or the precursor poly(acrylic acid-co-styrene) copolymer has the structure of formula (I-P): 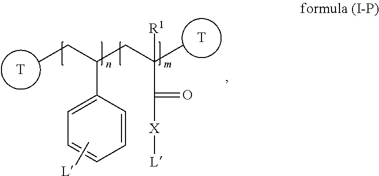
wherein each L′ is independently selected from H and a reactive handle, and
T, n, m, R¹, and X are as set out in any one of claims 6 to 11 or 27 to 28.

36. The method according to any one of claims 34 and 35, wherein each L′ is independently selected from the group consisting of H, CH₂Cl, CH₂Br, or CH₂I.

37. The copolymer-drug-conjugate according to any one of claims 1 to 30 or the pharmaceutical composition according to any one of claims 31 to 32, for use in medicine.

38. The copolymer-drug-conjugate according to any one of claims 1 to 30 or the pharmaceutical composition according to any one of claims 31 to 32, for use in treatment of cancer.

39. The copolymer-drug-conjugate or the pharmaceutical composition for use according to claim 38, wherein the cancer is characterised by the presence of a tumour.

40. The copolymer-drug-conjugate or the pharmaceutical composition for use according to claim 39, wherein the tumour is a solid tumour.

41. The copolymer-drug-conjugate or the pharmaceutical composition for use according to claim 39 or 40, wherein the tumour such as the solid tumour is a sarcoma.

42. The copolymer-drug-conjugate or the pharmaceutical composition for use according to claim 39 or 40, wherein the tumour such as the solid tumour is a carcinoma.

43. The copolymer-drug-conjugate or the pharmaceutical composition for use according to any one of claims 40 to 42, wherein the solid tumour is a primary or a metastatic tumour.

44. The copolymer-drug-conjugate or the pharmaceutical composition for use according to any one of claims 39 to 43, wherein the cancer is liver cancer or spleen cancer.

45. The copolymer-drug-conjugate or the pharmaceutical composition for use according to any claims 38 to 44, wherein the copolymer-drug-conjugate or the pharmaceutical composition is administered intratumourally, such as by injection.

46. A method of treating cancer, such as a cancer characterised by a tumour, such as a solid tumour, said method comprising administering the copolymer-drug-conjugate according to any one of claims 1 to 30 or the pharmaceutical composition according to any one of claims 31 to 32 to a subject in need thereof.

47. A method of inducing an immune response in a subject, said method comprising administering the copolymer-drug-conjugate according to any one of claims 1 to 30 or the pharmaceutical composition according to any one of claims 31 to 32 to said subject.

48. A method of immunising a subject, said method comprising administering the copolymer-drug-conjugate according to any one of claims 1 to 30 or the pharmaceutical composition according to any one of claims 31 to 32 to said subject.

49. A method of reducing tumour volume in a subject, said method comprising administering the copolymer-drug-conjugate according to any one of claims 1 to 30 or the pharmaceutical composition according to any one of claims 31 to 32 to said subject.

50. Use of the copolymer-drug-conjugate according to any one of claims 1 to 30 or the pharmaceutical composition according to any one of claims 31 to 32 as an immunostimulator.

51. Use of the copolymer-drug-conjugate according to any one of claims 1 to 30 or the pharmaceutical composition according to any one of claims 31 to 32 for the manufacture of a medicament for use in the treatment of cancer.

52. Use of a copolymer polymerized from:
a. styrene and/or diisobutylene, and
b. acrylic acid, methacrylic acid, and/or maleic acid
for retaining an agent within a tissue.

53. The use according to claim 52, wherein the copolymer is a poly(acrylic acid-co-styrene) copolymer, a poly(methacrylic acid-co-styrene) copolymer, a poly(maleic acid-co-styrene) copolymer, or a poly(diisobutylene-co-maleic acid) copolymer.

54. A method of increasing the residence time of an agent in a tissue such as a cancer tissue, said method comprising administering the agent together with a copolymer polymerized from:
a. styrene and/or diisobutylene, and
b. acrylic acid, methacrylic acid, and/or maleic acid.

55. The method according to claim 54, wherein the copolymer is a poly(acrylic acid-co-styrene) copolymer, a poly(methacrylic acid-co-styrene) copolymer, a poly(maleic acid-co-styrene) copolymer, or a poly(diisobutylene-co-maleic acid) copolymer.
